(19) United States
(12) Patent Application Publication
(10) Pub. No.: US 2025/0107882 A1
(43) Pub. Date: Apr. 3, 2025

(54) DEVICES FOR AORTIC REPAIR, AND ASSOCIATED SYSTEMS AND METHODS

(71) Applicant: Mavericks Endo, Inc., Menlo Park, CA (US)

(72) Inventors: G Ray Martin, Redwood City, CA (US); Anuja Harshadray Patel, Pacifica, CA (US); Richard Roy Newhauser, Belmont, CA (US); Morgan Alex Jawitz, Mountain View, CA (US); Eric Anthony Kramer, Los Altos Hills, CA (US); Wendy Ngo Lam, San Jose, CA (US)

(21) Appl. No.: 18/793,715

(22) Filed: Aug. 2, 2024

Related U.S. Application Data
(60) Provisional application No. 63/530,420, filed on Aug. 2, 2023.

(51) Int. Cl. A61F 2/07; A61F 2/06

(52) U.S. Cl. CPC A61F 2/07; A61F 2002/061

(57) ABSTRACT

Aortic repair devices and associated systems and methods are disclosed herein. An aortic repair device configured in accordance with embodiments of the present technology can include a base member configured to be implanted in an aorta proximate to a diseased portion of the aorta, such as an aneurysm or dissection near the aortic arch. The base member can direct blood flow past the diseased portion of the aorta to perfuse a portion of the aorta distal of the diseased portion. The aortic repair device can further include a leg member configured to be coupled to the base member. The leg member can provide a flow path from the base member to a branch vessel.

### CROSS-REFERENCE TO RELATED APPLICATION(S)

The present application claims priority to U.S. Provisional App. No. 63/530,420, filed Aug. 2, 2023, the entirety of which is hereby incorporated by reference.

### TECHNICAL FIELD

The present technology is directed to devices, systems, and methods for repairing a diseased aorta, and more particularly to devices configured to be implanted at least partially within the proximal aorta to treat aneurysms and dissections in the ascending aorta and/or the aortic arch.

### BACKGROUND

Aneurysms, dissections, penetrating ulcers, intramural hematomas, and/or transections may occur in blood vessels, and most typically occur in the aorta and peripheral arteries. A diseased region of the aorta may extend into areas having vessel bifurcations or segments of the aorta from which smaller “branch” arteries extend.

The diseased region of the aorta and other vessels can be bypassed with a stent graft placed inside the vessel to span the diseased region. The stent graft can effectively seal off the diseased region from further exposure to blood flow, inhibiting or preventing the aneurysm, dissection, or other type of diseased region from worsening. However, the use of stent grafts to internally bypass a diseased region of a vessel is not without challenges. In particular, care must be taken so that the stent graft does not cover or occlude critical branch vessels, yet the stent graft must adequately seal against the healthy regions of the vessel wall and remain open to provide a flow conduit for blood to flow past the diseased region.

### BRIEF DESCRIPTION OF THE DRAWINGS

Many aspects of the present disclosure can be better understood with reference to the following drawings. The components in the drawings are not necessarily to scale. Instead, emphasis is placed on clearly illustrating the principles of the present disclosure.

FIGS. 1A and 1B are side views of a diseased aorta and surrounding anatomy in which an aortic repair device can be implanted by a delivery system in accordance with embodiments of the present technology.

FIGS. 2A-2D are side cross-sectional views of a diseased aorta in which an aortic repair device can be implanted by a delivery system in accordance with embodiments of the present technology.

FIGS. 3A and 3B are perspective side views of a base member of an aortic repair device configured in accordance with embodiments of the present technology.

FIG. 3C is a perspective view of a first end portion of the base member of the aortic repair device of FIGS. 3A and 3B, and FIG. 3D is a schematic cross-sectional view of the base member of the aortic repair device of FIGS. 3A and 3B taken along the line 3D-3D in FIG. 3A.

FIG. 3E is a is a perspective end-on view of the first end portion of the base member of the aortic repair device of FIGS. 3A and 3B in accordance with additional embodiments of the present technology.

FIG. 3F is a side view of a stent (shown in a flattened state) of a base member of an aortic repair device configured in accordance with embodiments of the present technology.

FIGS. 4A-4C are perspective side views of a leg member of an aortic repair device in accordance with embodiments of the present technology.

FIG. 4D is a perspective view of a first end portion of the leg member of the aortic repair device of FIGS. 4A-4C.

FIG. 4E is a top view of transition stents of the leg member of FIGS. 4A-4C.

FIG. 5A is a perspective view of an aortic repair device including the base member of FIGS. 3A-3D coupled to the leg member of FIGS. 4A-4D in accordance with embodiments of the present technology.

FIG. 5B is a perspective view of a first end portion of the aortic repair device of FIG. 5A.

FIGS. 6A-6C are perspective views of the aortic repair device of FIGS. 5A and 5B with the leg member inserted at different positions within a secondary lumen of the base member in accordance with embodiments of the present technology.

FIGS. 7A-7C are images of various stages of a procedure to implant the base member of FIGS. 3A-3D within a model of an aorta of a patient in accordance with embodiments of the present technology.

FIG. 8A is a perspective side view of a distal portion of a delivery system secured to the leg member of FIGS. 4A-4D in accordance with embodiments of the present technology.

FIG. 8B is an enlarged view of an end portion of the leg member and a leading tip capture mechanism of the delivery system of FIG. 8A in accordance with embodiments of the present technology.

FIGS. 9A-9D are images of various stages of a procedure for deploying the leg member of FIGS. 4A-4D, 8A, and 8B within a model of an aorta of a patient and coupling the leg member to the base member of FIGS. 3A-3D and 7A-7C in accordance with embodiments of the present technology.

FIGS. 10A and 10B are side views of different stages of deploying the leg member of FIGS. 4A-4D with a delivery system in accordance with additional embodiments of the present technology.

FIG. 11 is a side view of the base member of the aortic repair device of FIGS. 3A-3D implanted within an aorta after implantation via the delivery system of FIGS. 7A-7C in accordance with embodiments of the present technology.

FIG. 12 is a side view of the aortic repair device of FIG. 11 with a leg member of FIGS. 4A-4D coupled to the base member in accordance with embodiments of the present technology.

FIG. 13 is a side view of an aortic repair device implanted within an aorta in accordance with additional embodiments of the present technology.

FIG. 14 is a side view of an aortic repair device implanted within an aorta in accordance with additional embodiments of the present technology.

### DETAILED DESCRIPTION

The present technology is directed to devices for treating a diseased aorta and/or other vessels of a patient, such as a human patient, and associated systems and methods. In some embodiments, for example, a repair device includes a tubular base member configured to be implanted in a proximal region of a vessel (e.g., an aorta) proximate to a diseased portion of the vessel, such as an aneurysm or dissection. The base member can provide a conduit for directing blood flow past the diseased portion of the vessel to perfuse a portion of the vessel distal of the diseased portion. In some embodiments, the repair device can further include a leg member configured to be coupled to the base member. The leg member can provide a flow path from the base member in the main vessel to a branch vessel.

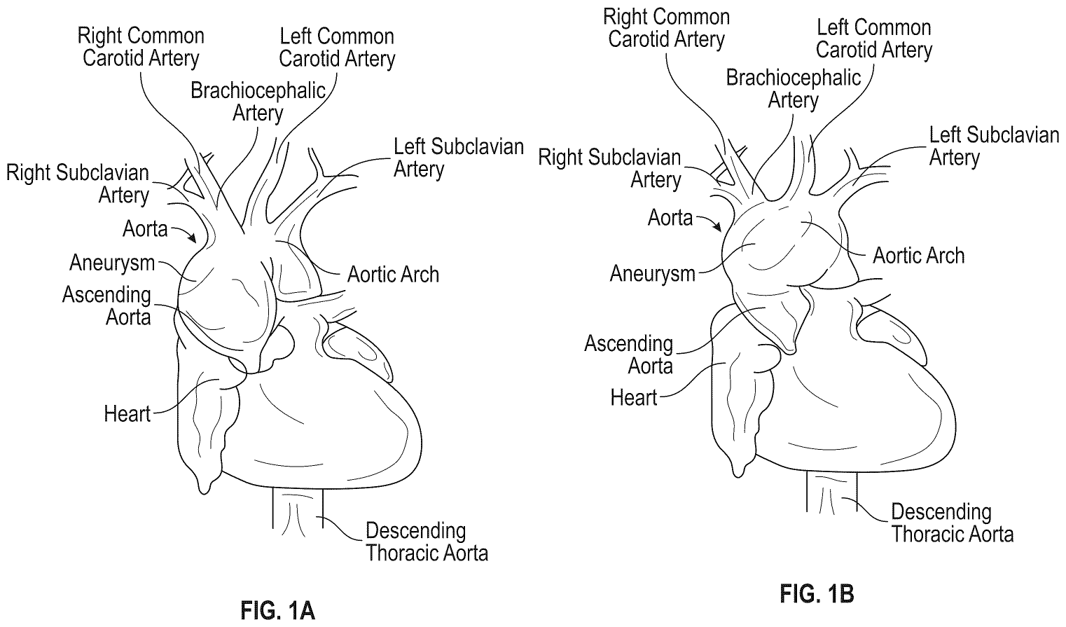

Specific details of several embodiments of the present technology are described herein with reference to FIGS. 1A-14. The present technology, however, can be practiced without some of these specific details. In some instances, well-known structures and techniques often associated with catheter-based delivery systems, implantable repair devices, and the like, have not been shown in detail so as not to obscure the present technology. Although many of the embodiments are described below with respect to medical devices, systems, and methods for treatment of an aneurysm and/or a dissection of an aorta positioned along or near the aortic arch, other applications and other embodiments in addition to those described herein are within the scope of the technology. For example, the present technology may be used for the treatment of different portions of the aorta (e.g., the abdominal aorta), other vessels (e.g., vessels having significant curvature and/or branching vessels near the diseased portion), and/or different indications. Additionally, several other embodiments of the technology can have different configurations, components, or procedures than those described herein, and that features of the embodiments shown can be combined with one another. A person of ordinary skill in the art, therefore, will accordingly understand that the technology can have other embodiments with additional elements, or the technology can have other embodiments without several of the features shown and described below with reference to the Figures.

The terminology used in the description presented below is intended to be interpreted in its broadest reasonable manner, even though it is being used in conjunction with a detailed description of certain specific embodiments of the disclosure. Certain terms can even be emphasized below; however, any terminology intended to be interpreted in any restricted manner will be overtly and specifically defined as such in this Detailed Description section.

The accompanying Figures depict embodiments of the present technology and are not intended to be limiting of its scope. The sizes of various depicted elements are not necessarily drawn to scale, and these various elements can be arbitrarily enlarged to improve legibility. Component details can be abstracted in the Figures to exclude details such as position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the spirit or scope of the present technology.

When used with reference to an aortic repair device, the term “distal” can reference a portion of the aortic repair device positioned and/or configured to be positioned farther from the heart and downstream in the path of blood flow, while the term “proximal” can reference a portion of the aortic repair device positioned and/or configured to be positioned closer to the heart and upstream in the path of blood flow. In contrast, when used with reference to a catheter subsystem or delivery procedure, the term “distal” can reference a portion of the catheter system farther from an operator and/or handle while the term “proximal” can reference a portion of the catheter system closer to the operator and/or handle. Accordingly, as set forth below, often a proximal portion of an aortic repair device is farther from the operator and/or handle of a catheter system used to deliver the aortic repair device than a distal portion of the aortic repair device. Likewise, often a distal portion of the aortic repair device is closer to the operator and/or handle of the catheter system than the proximal portion.

Also, as used herein, the designations “rearward,” “forward,” “upward,” “downward,” etc., are not meant to limit the referenced component to use in a specific orientation. It will be appreciated that such designations refer to the orientation of the referenced component as illustrated in the Figures; the systems of the present technology can be used in any orientation suitable to the user.

The headings provided herein are for convenience only and should not be construed as limiting the subject matter disclosed.

### I. Overview

FIGS. 1A and 1B are side views of a diseased aorta and surrounding anatomy in which a medical device, such as an aortic repair device, can be implanted by a delivery system in accordance with embodiments of the present technology. Referring to FIGS. 1A and 1B together, the aorta is the largest vessel in the human body and carries oxygenated blood away from the left ventricle and the aortic valve of the heart for circulation to all parts of the body. The aorta is divided into different segments including the ascending aorta (which extends from the left ventricle), the aortic arch, and the descending thoracic aorta. The ascending aorta and the aortic arch can together be referred to as the “proximal aorta.” The aorta includes branches into several supra-aortic arteries including the brachiocephalic artery, the left common carotid artery, and the left subclavian artery—each of which extends from the aortic arch. The brachiocephalic artery is the first branch of aortic arch and feeds blood flow to the right common carotid artery and the right subclavian artery for supply to the right arm, head, and neck. It is also known as the innominate artery or the brachiocephalic trunk. The left common carotid artery is the second branch of the aortic arch and feeds blood flow to the left head and neck. The left subclavian artery is the third branch of the aortic arch and feeds blood flow to the left arm.

The diseased aorta includes an aneurysm in the ascending aorta in FIG. 1A and an aneurysm in the aortic arch in FIG. 1B. Aortic aneurysms are enlargements (e.g., dilations) of the aorta that weaken the aorta and increase the likelihood of rupture. Most people with aortic aneurysms do not have symptoms until the aorta ruptures, and the mortality rate after an aortic aneurysm rupture can exceed 90%. Often, aortic aneurysms are detected during routine medical testing such as chest X-rays, computed tomography (CT) imaging, ultrasound imaging of the heart, magnetic resonance imaging (MRI), and the like.

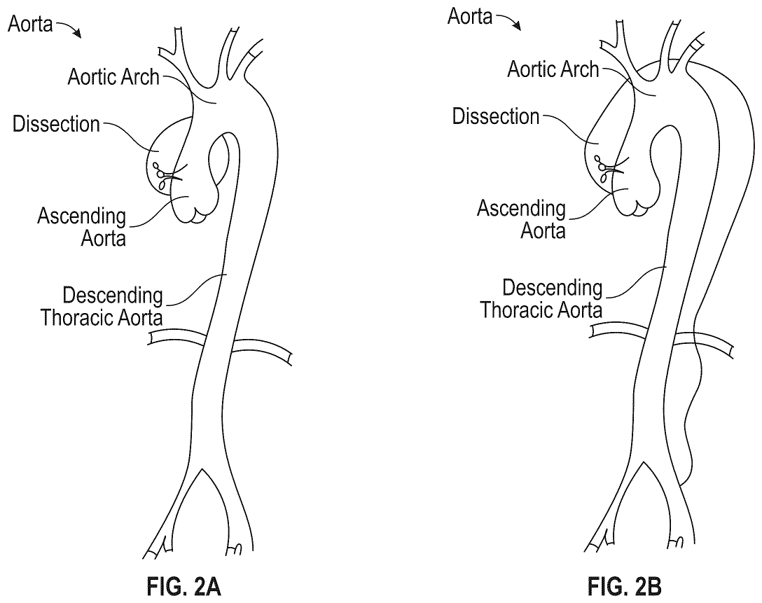

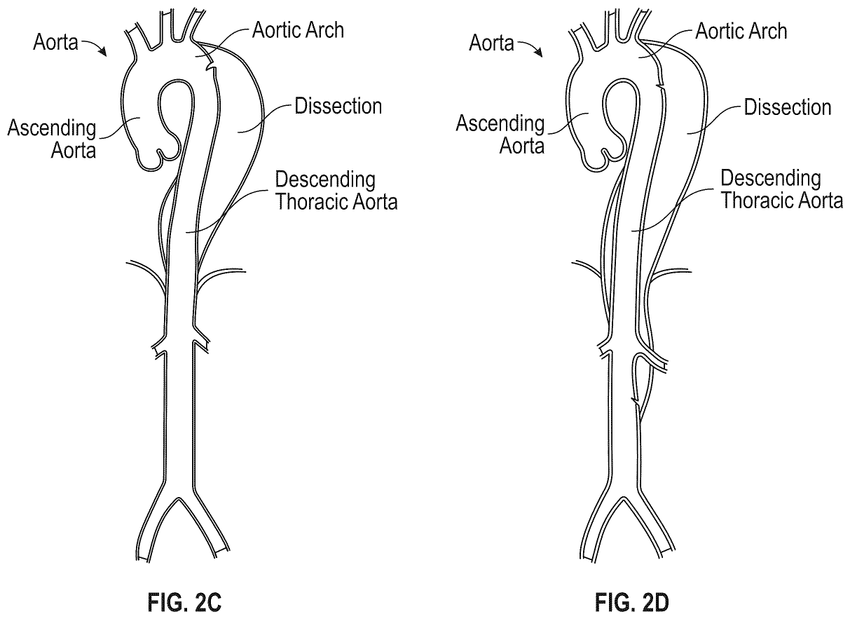

FIGS. 2A-D are side cross-sectional views of a diseased aorta in which a medical device, such as an aortic repair device, can be implanted by a delivery system in accordance with embodiments of the present technology. The diseased aorta includes DeBakey Type II, a smaller Type A aortic dissection contained within the ascending aorta, in FIG. 2A, and a DeBakey Type I, a larger Type A aortic dissection that extends beyond the ascending aorta, in FIG. 2B. The diseased aorta includes a smaller Type B aortic dissection in FIG. 2C, and a larger Type B aortic dissection in FIG. 2D. Aortic dissections are tears of the intimal layer of the aorta that cause blood to dissect the intimal layer and block flow. Type A aortic dissections originate in the ascending aorta and can progressively extend from the ascending aorta, along the aortic arch, and/or down along the descending aorta as shown in FIG. 2B. Type B aortic dissections originate distal to the branching of the brachiocephalic trunk and can progressively extend down along the descending aorta as shown in FIG. 2D. Aortic dissections can be acute or chronic, with acute aortic dissections frequently causing a sudden onset of severe pain in the chest, back, and/or abdomen. Most acute aortic dissections require emergency surgery, and present about a 1% risk of death every hour for the first two days after dissection-meaning that urgent diagnosis and surgery are critical for patient treatment.

Referring to FIGS. 1A-2D together, aspects of the present technology are directed to medical device, such as aortic repair devices, that can be implanted within the aorta to treat an aortic aneurysm, a Type A aortic dissection, a Type B aortic dissection, and/or other types of diseased states. In some embodiments, an aortic repair device can span across the origin of an aneurysm or dissection and provide one or more flow conduits for diverting blood flow away from and/or past the diseased portion. In some embodiments, an aortic repair device can span between and provide one or more flow conduits for directing blood flow between the aorta and one or more branching arteries.

### II. Selected Embodiments of Aortic Repair Devices

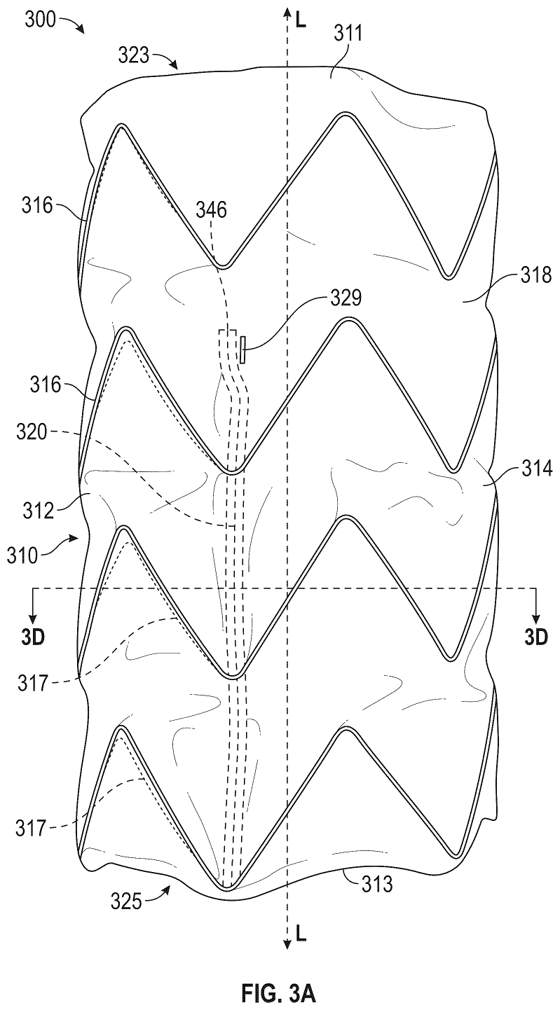

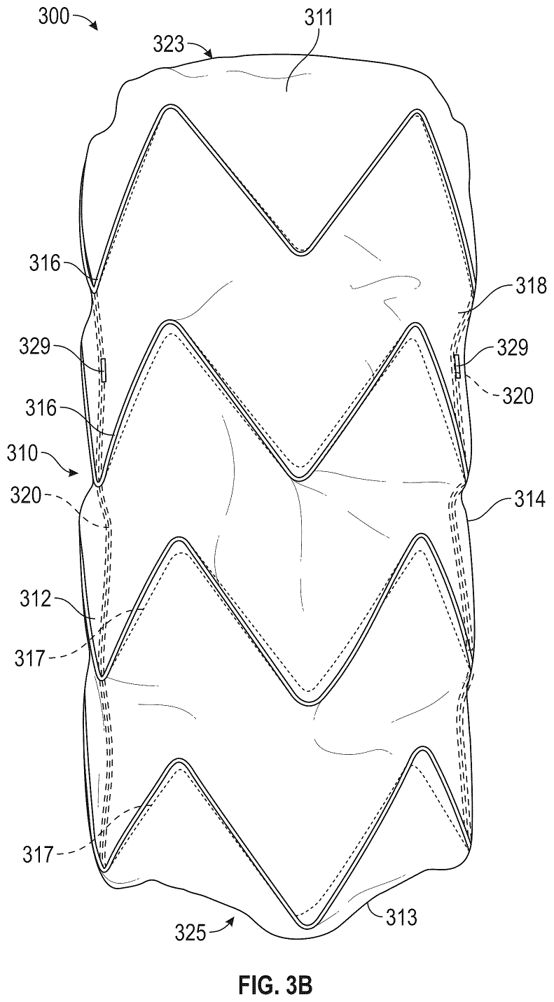

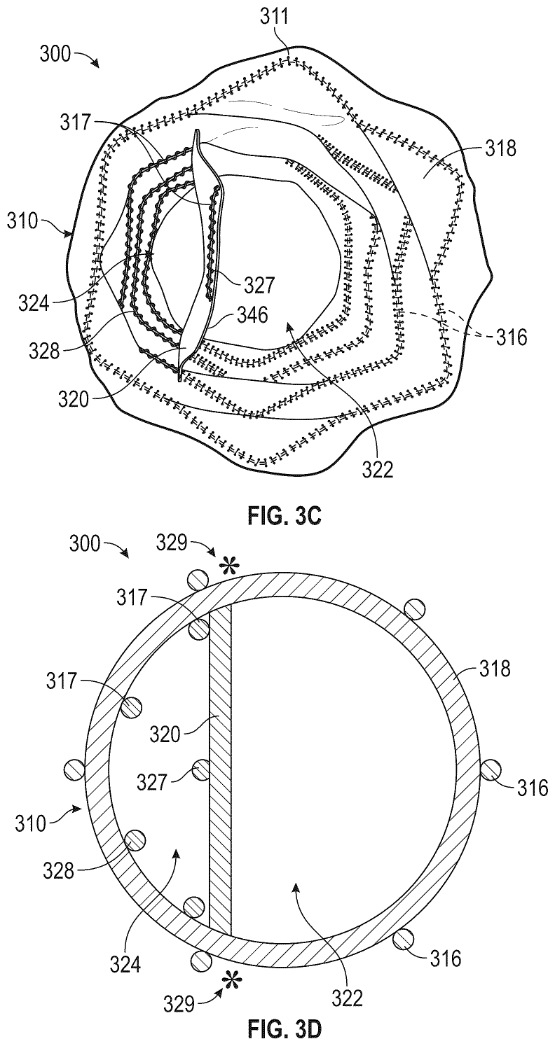

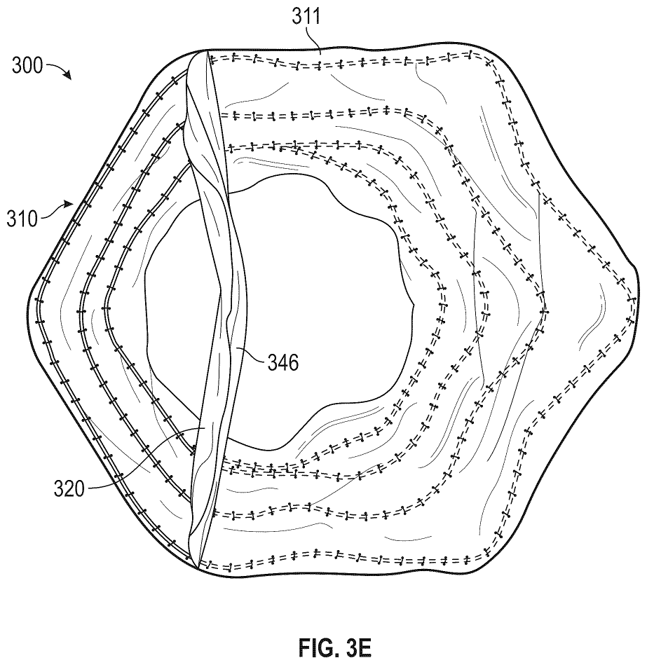

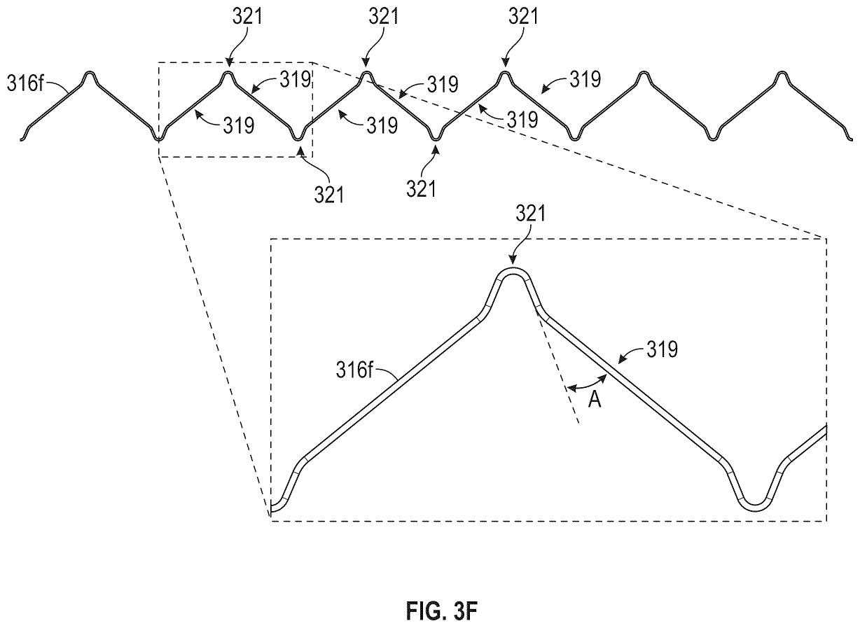

FIGS. 3A and 3B are perspective side views of a base member 310 of an aortic repair device 300 (which can also be referred to as an aortic arch repair device, an aortic prosthesis, an aortic treatment device, an aortic implant, a repair device, a medical device, and/or the like) in accordance with embodiments of the present technology. The base member 310 is rotated counterclockwise by 90 degrees in FIG. 3B relative to the view in FIG. 3A. FIG. 3C is a perspective view of the leading end portion 311 of the base member 310 of the aortic repair device 300 of FIGS. 3A and 3B, and FIG. 3D is a schematic cross-sectional view of the base member 310 of the aortic repair device 300 of FIGS. 3A and 3B taken along the line 3D-3D in FIG. 3A. The aortic repair device 300 can include some features that are at least generally similar in structure and function, or identical in structure and function, to any of the aortic repair devices disclosed in U.S. patent application Ser. No. 18/179,254, Filed Mar. 6, 2023, and titled “DEVICES FOR AORTIC REPAIR, AND ASSOCIATED SYSTEMS AND METHODS,” which is incorporated by reference herein in its entirety.

Referring to FIGS. 3A and 3B, the base member 310 is configured to be implanted in a diseased aorta and includes a leading (e.g., first, proximal) end portion 311 defining a leading (e.g., first, leading) terminus of the base member 310 and a trailing (e.g., second, distal) end portion 313 defining a trailing terminus of the base member 310. The base member 310 further includes (i) a first side portion 312 that is configured to be implanted and positioned adjacent/proximate to an outer portion (e.g., an outer curved portion) of an interior wall of an aorta having a greater curvature (e.g., greater radius of curvature, longer length), and (ii) a second side portion 314 opposite to the first side portion 312 that is configured to conform to an inner portion (e.g., an inner curved portion) of the interior wall of the aorta having a lesser curvature (e.g., a smaller radius of curvature, shorter length). The base member 310 can be generally hollow to define a channel through which blood can flow from a leading opening 323 (e.g., a fluid opening, a first opening, a first fluid opening, an inlet, and/or the like) to a trailing opening 325 (e.g., a fluid opening, a second opening, a second fluid opening, a distal outlet, and/or the like). A diameter of the base member 310 can be sized to generally match or be larger than (e.g., oversized relative to) the diameter of a patient's aorta (e.g., between about 20-60 millimeters, between about 26-54 millimeters, about 40 millimeters).

The base member 310 can be an expandable stent graft that includes one or more stents (identified as “first stents 316” and “second stents 317”) coupled to an inner or outer surface of a graft material 318 via stitching (e.g., sutures) and/or other suitable attachment techniques. As shown in FIGS. 3A and 3B, the first and second stents 316, 317 can have a V-pattern shape (e.g., including alternating leading and trailing apices). In some embodiments, the stents 316, 317 may have other suitable geometries, such as Z-stents, braided stents, and/or other configurations. In the embodiment illustrated in FIGS. 3A-3D, the first stents 316 are secured to an outer surface of the graft material 318 and extend circumferentially (e.g., circularly) about the outer surface of the graft material 318, and the second stents 317 are secured to an inner surface of the graft material 318 (and thus illustrated using dashed line and/or including their attachment points (e.g., stitching) to the graft material 318). Accordingly, the first stents 316 can be referred to as abluminal stents and the second stents 317 can be referred to as intraluminal stents. The first and second stents 316, 317 can be configured to self-expand and, accordingly, can be formed from a shape memory material, such as nickel-titanium alloy (nitinol). The graft material 318 can comprise fabric, woven polyester, polytetrafluoroethylene, polyurethane, silicone, and/or other suitable materials known in the art of stent grafts, and is configured to inhibit or even prevent blood flow therethrough. In some embodiments, the first stents 316 (i.e., outer stents) can have a diameter in the expanded state that is larger than the diameter of the graft material 318 to which it is attached.

Referring to FIGS. 3C and 3D, the base member 310 can include a septum 320 (e.g., a flow divider, a wall, a barrier) positioned therein to divide the channel of the base member 310 into a primary lumen 322 (e.g., a first lumen) and a branch or secondary lumen 324 (e.g., a second lumen). The primary lumen 322 and the secondary lumen 324 can each have a D-like cross-sectional shape within the base member 310 as the septum 320 bifurcates the lumen defined by the base member 310. In some embodiments, the septum 320 can have a different shape or structure to divide the base member 310 into lumens having circular cross-sectional shapes, crescent shapes, and/or other shapes, and/or the septum 320 may define first and second lumens having different shapes. In the illustrated embodiment, the septum 320 is offset within the base member 310 such that the primary lumen 322 is larger than the secondary lumen 324. For example, the secondary lumen 324 can account for up to 5%, 10%, 15%, 20%, 25%, or 30% of a cross-sectional area of the base member 310 and the primary lumen 322 can account for at least 70%, 75%, 80%, 85%, 90%, or 95% of the cross-sectional area. In other embodiments, the septum 320 can be centered within the base member 310 such that the primary and secondary lumens 322, 324 have the same size, or the septum 320 can be offset within the base member 310 such that the secondary lumen 324 is larger than the primary lumen 322. The septum 320 can be made from the same graft material 318 as the base member 310, another type of graft material, and/or another type of liquid impermeable material. Accordingly, the graft material 318 and the septum 320 can define and enclose the primary lumen 322 and the secondary lumen 324 and are configured to maintain blood flowing along the separate flow paths defined thereby. As shown in FIGS. 3A-3D, the primary lumen 322 can extend along the second side portion 314 of the base member 310 and the secondary lumen can extend along the first side portion 312 of the base member 310.

Referring to FIGS. 3A-3D, fluid (e.g., blood) flowing into the leading opening 323 of the base member 310 is divided/diverted into the primary lumen 322 or the secondary lumen 324 and routed to the trailing body opening 325. In some embodiments, the septum 320 extends only partially between the leading and trailing end portions 311, 313. For example, in the illustrated embodiment (as best seen in FIG. 3C), the septum 320 extends from the trailing end portion 313 only partially toward the leading end portion 311 such that septum 320 includes a leading end portion 346 positioned within the base member 310. Accordingly, the septum 320 divides the trailing opening 325 into a first trailing opening and a second trailing opening. In other embodiments, the septum 320 extends along the entire length of the base member 310 between the leading and trailing end portions 311, 313 such that the septum 320 divides the trailing opening 325 into a first trailing opening and a second trailing opening and divides the leading opening 323 into a first leading opening and a second leading opening. For example, FIG. 3E is a perspective end-on view of the leading end portion 311 of the base member 310 of the aortic repair device 300 of FIGS. 3A and 3B in accordance with additional embodiments of the present technology. In the illustrated embodiment, the septum 320 extends entirely to the leading end portion 311 of the base member 310 such that the leading end portion 346 of the septum 320 is positioned at and/or adjacent to the leading end portion 311 of the base member 310.

Referring again to FIGS. 3C and 3D, the first stents 316 extend entirely about the outer surface of the graft material 318 and can have a circular cross-sectional shape. Accordingly, none of the first stents 316 are attached to or define the septum 320, nor do the first stents 316 abut or extend along a portion of the septum 320. In the illustrated embodiment, the second stents 317 are positioned within the secondary lumen 324 and can be attached to or at least extend about a portion of the inner surface of the graft material 318 extending around the base member 310 and the septum 320. Accordingly, the second stents 317 can be referred to as secondary lumen stents, intraluminal stents, and/or the like. In some embodiments, the second stents 317 have a D-like cross-sectional shape that matches the D-like cross-sectional shape of the secondary lumen 324. More specifically, the second stents 317 can each have a generally flat or planar portion 327 (also referred to as inner portion 327; certain embodiments having a bowed shape projecting into the primary lumen 322) and a generally curved portion 328 (also referred to as an outer portion 328; optionally matching the curvature of the first stents 316). The flat portions 327 of the second stents 317 can be positioned adjacent to and/or coupled to the septum 320 and the curved portions 328 of second stents 317 can be positioned adjacent to and/or coupled to the inner surface of the graft material 318. In other embodiments, the second stents 317 can have other cross-sectional shapes, such as circular, elliptical, semicircular, bowed D-like, etc. For example, the flat portions 327 of the second stents 317 can be bowed outward toward the primary lumen 322.

The flat portions 327 and the curved portions 328 of the second stents 317 can define respective arc lengths. In some embodiments, the arc length of the flat portion 327 and the arc length of the curved portions 327, 328 can each be at least generally similar or identical across base members of differing sizes (e.g., base members having outer diameters from an about 37 mm to about 40 mm) such that the secondary lumen 324 has a similar or identical cross-sectional area across differently sized base members. Accordingly, the flat and curved portions 327, 328 can define secondary lumens that occupy a larger portion of the cross-sectional area in smaller base member sizes (e.g., a secondary lumen occupying 24% of the cross-sectional area of a 37 mm base member) relative to that of larger base member sizes (e.g., a secondary lumen occupying 20% of the cross-sectional area of a 40 mm base member). In various embodiments, the secondary lumen 324, as defined by the secondary stents 317, can occupy a cross-sectional area that varies from 20% to 25% of the overall cross-sectional lumen area of the base member 310 (depending upon the diameter of the base member), while still maintaining the patency of both primary and secondary lumens 322, 324 and providing for appropriate sealing of a leg component positioned within the secondary lumen 324. With the size of the secondary lumen 324 remaining substantially the same across differently sized base members (e.g., sized to fit into differently sized aortas), the same components (e.g., the secondary stents 317) can be used in the manufacture of differently sized base members and the same leg component can be deployed within base members of different sizes because the cross-sectional area of the secondary lumen 324 remains the same, thereby eliminating the need for unique leg members for differently sized base members.

Further, in embodiments in which a spanning member is deployed within the primary lumen 322, the same spanning member can be oversized in a manner that allows the same spanning manner to be deployed within base members of various different sizes (e.g., 37 mm to 40 mm, greater than 40 mm, less than 37 mm). For example, the spanning member can have a diameter in the expanded/deployed state that is larger than that of the primary lumen 322 and thus fit within primary lumens 322 having differing cross-sectional areas. In some embodiments, a spanning member can fit within primary lumens 322 occupying 75% to 80% of the overall cross-section of the base member.

In some aspects of the present technology, the first stents 316 provide continuous circumferential support to the graft material 318 (which is configured to contact and seal against the inner surface of an aorta), while the second stents 317 provide continuous circumferential support within the secondary lumen 324. In some embodiments, the second stents are configured (e.g., formed of material, dimensioned) to be strong enough to maintain patency of the secondary lumen 324 when the base member 310 is implanted within an aorta without creating excessive loading force when constrained within a delivery catheter or excessive deployment force when deployed from the delivery catheter.

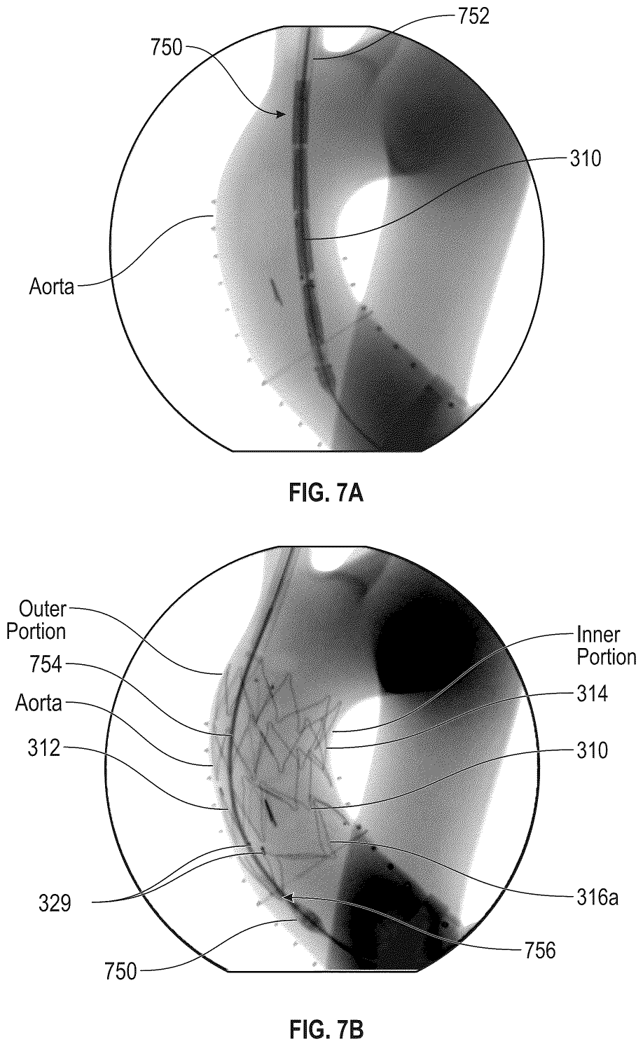

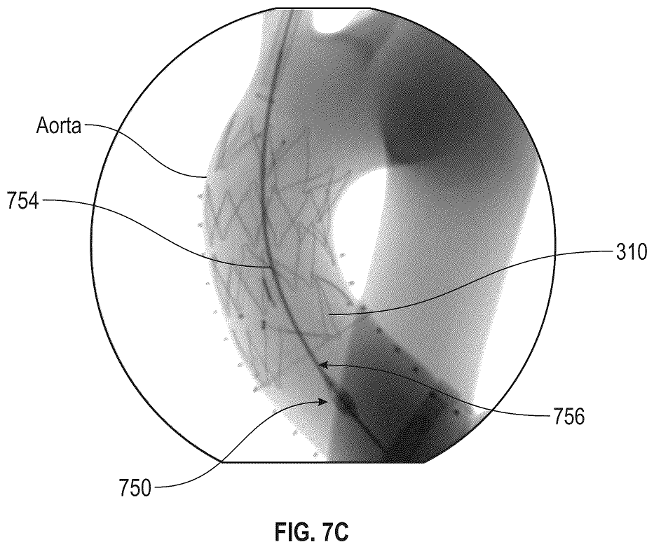

Referring to FIGS. 3A, 3B, and 3D, the base member 310 can further include one or more (e.g., two) radiopaque markers 329 to facilitate visualization of the base member 310 and is orientation during delivery of the base member 310 to an aorta and deployment therein. For example, as best seen in FIGS. 3B and 3D, the base member 310 can include a pair of the markers 329 positioned on opposing portions of the graft material 318, such as adjacent/proximate to opposing ends of the leading end portion 346 of the septum 320 where the leading end portion 346 meets the graft material 318. As described in greater detail below with reference to FIGS. 7A-7C, the markers 329 can facilitate rotational alignment of the base member 310 during delivery such that, for example, the first side portion 312 can be rotated into alignment with the outer portion of an interior wall of an aorta having a greater curvature.

As shown in FIGS. 3A and 3B, the first and second stents 316, 317 can have the same or substantially similar height along a longitudinal axis L (FIG. 3A) of the base member 310 and can have the same or a similar periodicity about the longitudinal axis L. In the illustrated embodiment, the first and second stents 316, 317 are aligned in phase with one another. In other embodiments, the first and seconds stents 316, 317 can be aligned out of phase with another and/or can have different heights, periodicities, configurations at the apices, shapes, and/or other features that differ from each other.

In some embodiments, one or more of the first stents 316 and/or the second stents 317 have non-linear patterns along their lengths and/or differing degrees of curvature at the apices of the stents 316, 317 to reduce strain on the stent, reduce the delivery profile of the device, and/or otherwise facilitate device delivery and deployment. For example, FIG. 3F is a side view (shown in a laid flat view) of a portion of a stent 316f for use with the base member 310 and/or other stent grafts disclosed herein. The stent 316f includes one or more first segments 319 that extend to one or more second segments 321 near the apices of the stent 316f. Each of the first segments 319 can be linear or at least generally linear. Each of the second segments 321 can have a curve, bend, U-shape, and/or other shape to define an apex portion positioned between and connecting individual ones of the first segments 319. As further shown in FIG. 3F, the second segments 321 can also be shaped (e.g., bent, laser cut) to define a non-zero angle A relative to the immediately adjacent first segments 319 (i.e., rather than extending directly along the linear path of the adjacent first segments 319). This forms an indent (also referred to as a dimple) as the stent 316f transitions from the first to the second segment 319, 321, and thereby defines a keyhole-like pattern at each apex. The angle A can range from about 1 degree to about 90 degrees, such as up to 10 degrees, 20 degrees, 30 degrees, 40 degrees, 45 degrees, 50 degrees, or another suitable angle. The location of the indent (i.e., where the angle A is formed) along the length of the first segment 319 and the extent of that indent can be selected to provide a desired strain profile and/or other features. In some embodiments, the second segment 321 can be formed in a manner such that it is out of plane with the adjoining first segments 319. For example, the second segment 321 or a portion thereof can be positioned inward of the first segments 319 (e.g., into the lumen formed by the stent 316f), positioned outward from the first segments 319 (e.g., directed outward toward an inner wall of the vessel in which it is positioned), or the plane may vary across different apices. The keyhole stent 316f can be formed using nitinol wire (e.g., rather than laser cut) using mandrels and/or other wire forming methods to impart the selected keyhole configuration.

The keyhole pattern of the stent 316f is expected to reduce strain on the stent 316f, reduce the delivery profile of the device 300, and reduce loading and deployment forces before and during delivery of the stent 316f to the target site. In at least some embodiments, for example, the keyhole-shaped second segments 321 allow the stent to assume a more compact delivery/loaded profile by, for example, allowing the apices to be positioned more compactly together than in a standard straight stent (reducing contact at the strut to apex interface) and/or allowing the first segments 319 to align with one another in a more parallel orientation and be positioned closer together when in the delivery/loaded profile. The keyhole pattern can also reduce the maximum crimp strain placed on the apices when the stent 316f is in the delivery profile as apices do not need to compress as much to attain the low-profile configuration. The keyhole shaped stent 316f can also reduce the peak radial force necessary for loading the stent 316f in a deliver system, in part because the stent 316f provides for self-packing (parallel strut alignment and reduction in contact of adjacent apices). This provides for a device that will be less resistant to loading into a smaller diameter delivery system, thereby casing the loading procedure and reducing the necessary force applied to the device during loading. Further, the enhanced stent packing of the keyhole stent 316f positions the apices in an orderly manner, keeping the interior space within the stent lumen free of overlapping struts and apices, and therefore increasing the volume within the stent 316f in which fabric (e.g., graft material or other covering) can be packed. Additionally, or alternatively, the decrease in overall force imparted on the stent 316f during loading means the stent 316f will also impart less force on the delivery system as it is exposed from the delivery sheath, thereby reducing jumping or other erratic behavior caused by the high force of expansion and providing for smooth, controlled delivery.

Furthermore, the keyhole stent 316f has been shown to maintain a high degree of radial resistive force (e.g., to support implantation and sealing to a vessel wall or the lumen of another device), which also provides enhanced anti-deformation properties in comparison to other standard stents. That is, the keyhole stent 316f configured in accordance with the present technology has been shown to have a radial resistive force that is similar to or higher than that of other known aortic stents on the market. However, unlike known stents, after the keyhole stent 316f has been loaded in the delivery system (e.g., via crimping into a delivery sheath, catheter, and/or other delivery component), the radial resistive force of the stent 316f is largely maintained after the stent 316f is re-expanded (e.g., as it would be at the target site). For example, keyhole stents 316f configured in accordance with the present technology have been shown to have a radial resistive force (with 5% oversizing) that decreases less than 7.6%, less than 3.9%, and less than 3.3% after loading. This is in comparison to other stents known in the industry that deform over 9.7%, over 11%, and over 15%. Thus, the keyhole stent 316f has less deformation post-loading and can maintain its radial resistive force to facilitate proper fixation and sealing to the adjoining vessel wall and/or a lumen of a mating device.

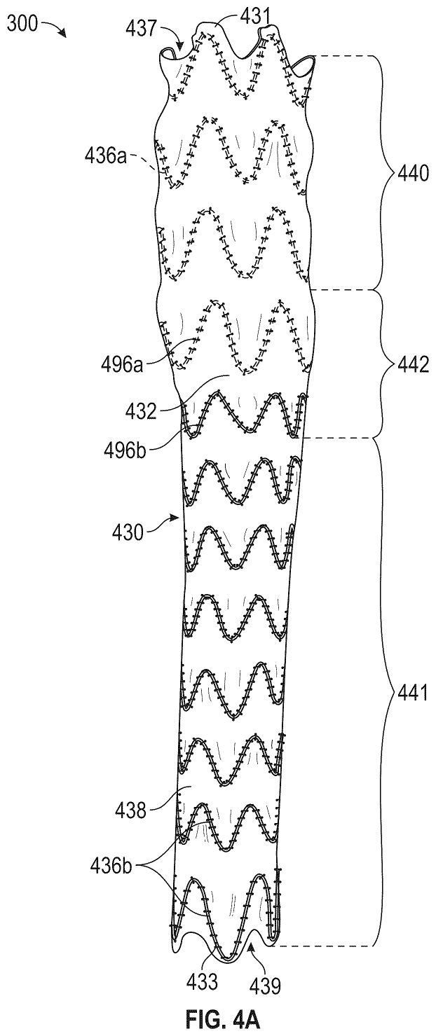

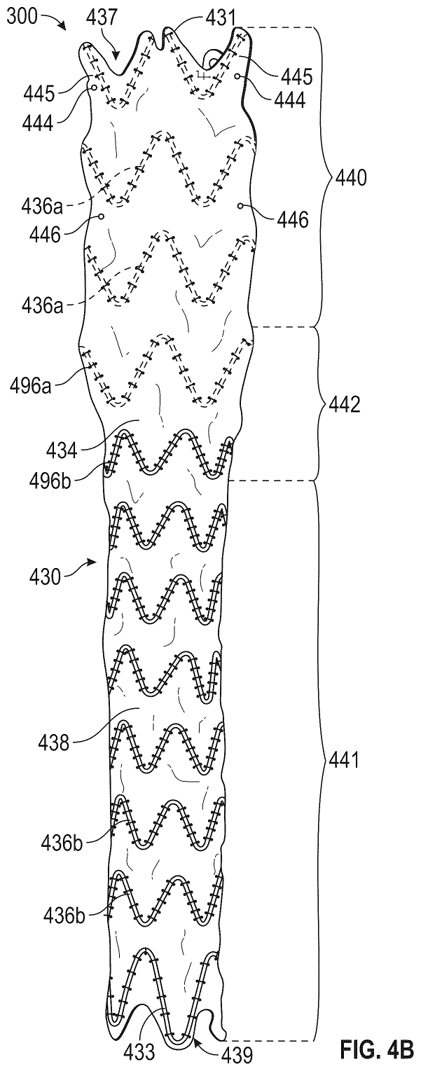

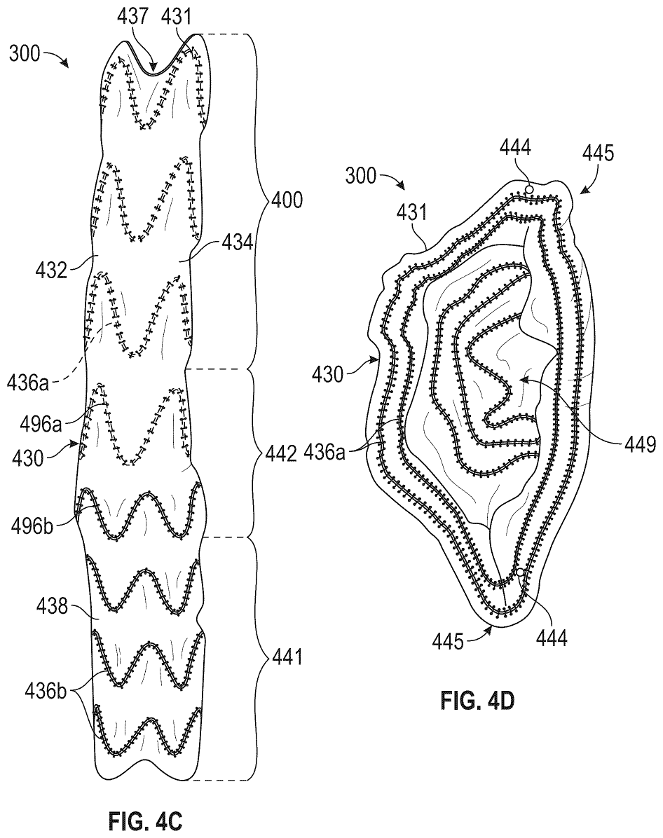

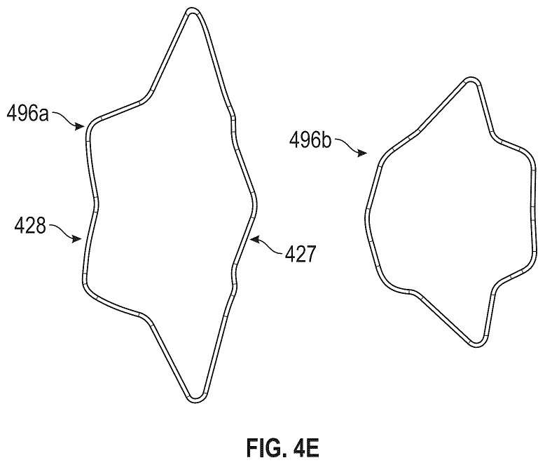

FIGS. 4A-4C are perspective side views of a leg member 430 (e.g., a branch member) of the aortic repair device 300 configured in accordance with embodiments of the present technology, and FIG. 4D is a perspective view of a leading end portion 431 of the leg member 430 of FIGS. 4A-4C. The leg member 430 is rotated by 180 degrees in FIG. 4B relative to the view in FIG. 4A, and is rotated by 90 degrees clockwise in FIG. 4C relative to FIG. 4A and by 90 degrees counterclockwise in FIG. 4C relative to FIG. 4B. Referring to FIGS. 4A-4C, the leg member 430 includes the leading end portion 431, a trailing end portion 433 (obscured in FIG. 4C), a first side portion 432 (obscured in FIG. 4B), and a second side portion 434 (obscured in FIG. 4A). The leg member 430 further includes a coupling portion 440 (e.g., first portion) that is at least partially configured to be received within the second lumen 324 of the base member 310 (FIGS. 3A-3D), a leg portion 441 (e.g., second portion, extension portion), and a transition portion 442 between the coupling portion 440 and the leg portion 441. The leg member 430 can be an expandable stent graft comprising a plurality of stents 436 (including individually identified first stents 436a and second stents 436b) coupled to a graft material 438. The stents 436 and the graft material 438 can be similar or identical in structure to the first and seconds stents 316, 317 and the graft material 318 described in detail above with reference to FIGS. 3A-3D. The leg member 430 is generally hollow and defines a leg lumen 449 (obscured in FIGS. 4A-4C). Referring to FIGS. 4A-4D the leg lumen 449 extends from a leading opening 437 (e.g., a fluid opening, a first opening, a first fluid opening, an inlet, and/or the like) at the leading end portion 431 of the leg member 430 to a trailing opening 439 (obscured in FIG. 4C; e.g., a fluid opening, a second opening, a second fluid opening, a distal outlet, and/or the like) at the trailing end portion 433 of the leg member 430.

As shown in FIGS. 4C and 4D, the first stents 436a can have a generally D-like cross-sectional shape (including a flat portion and a curved portion similar or identical to the flat portion 327 and the curved portion 328 described in detail above with reference to FIGS. 3C and 3D), and the second stents 436b can have a generally circular cross-sectional shape. Accordingly, the coupling portion 440 can have a D-like cross-sectional shape, the leg portion 441 can have a circular cross-sectional shape, and the transition portion 442 (including, e.g., any stents contained therein) can have a cross-sectional shape and/or outer perimeter that changes from D-like to circular in a direction from the leading end portion 431 toward the trailing end portion 433. For example, FIG. 4E is a top view of first and second transition stents 496a, 496b (“stents 496” or “transition stents 496”) included in the transition portion 442 of the leg member 430. Referring additionally to FIG. 4E, the transition stents 496 can have varying cross-sectional shapes and/or outer perimeters configured to transition the overall cross-sectional shapes and/or outer perimeter of the leg member 430 from, e.g., the D-like cross-sectional shape in the coupling portion 440 toward and/or to the circular cross-sectional shape in the leg portion 441. In the illustrated embodiment, for example, the first transition stent 496a is configured to be positioned proximate to the coupling 440 and the second transition stent 496b is configured to be positioned proximate to the leg portion 441. Accordingly, the first transition stent 496a includes a flat portion 427 and a curved portion 428 to define a generally D-like cross-sectional shape and the second transition stent 496b can have a generally circular shape.

In some embodiments, the first stents 436a, 496a can be attached to an inner surface of the graft material 438 within the leg lumen 449 and the second stents 436b, 496b can be attached to an outer surface of the graft material 438. In other embodiments, some or all of the stents 436, 496 can be attached to the graft material 438 in different arrangements (e.g., with some or all of the first stents 436a attached to the outer surface of the graft material 438, with some or all of the second stents 436b attached to the inner surface of the graft material 438, and so on).

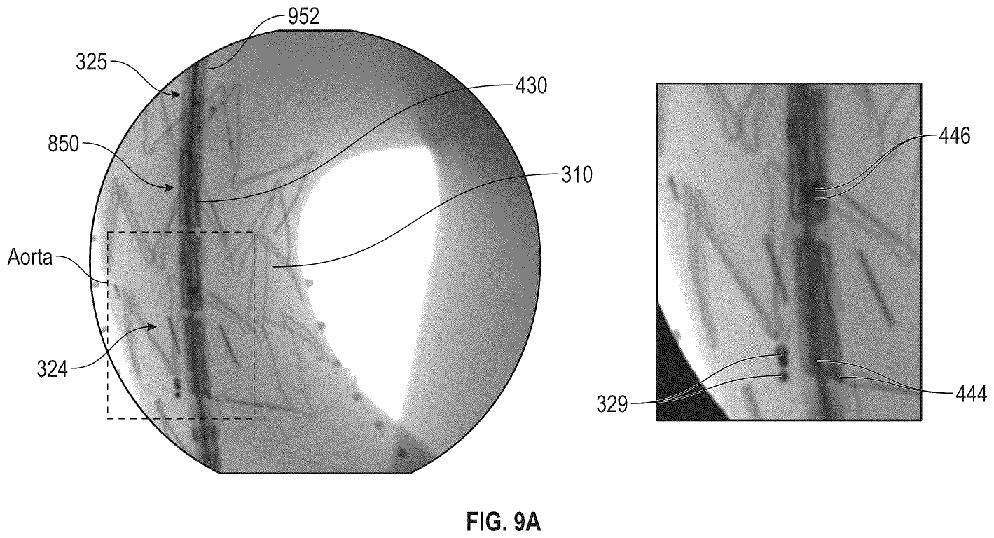

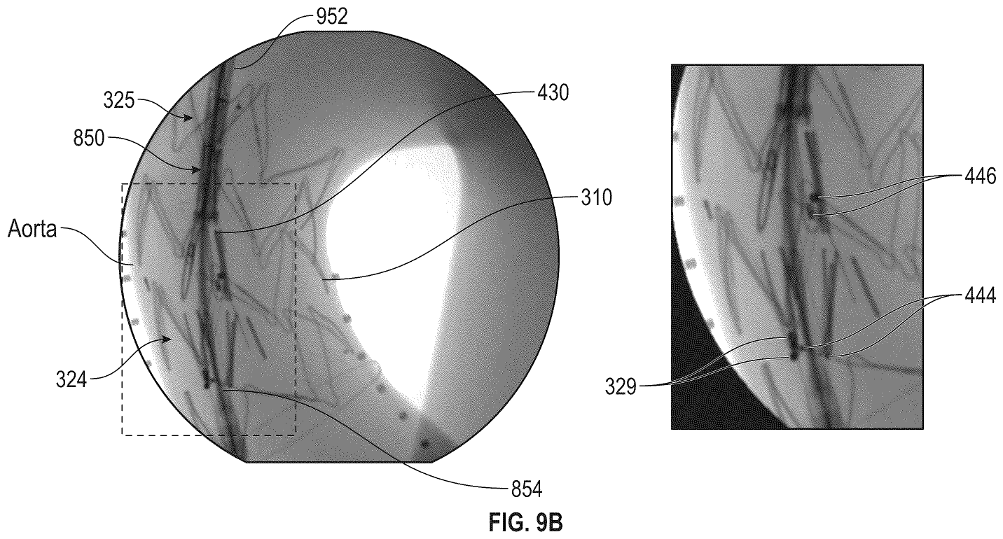

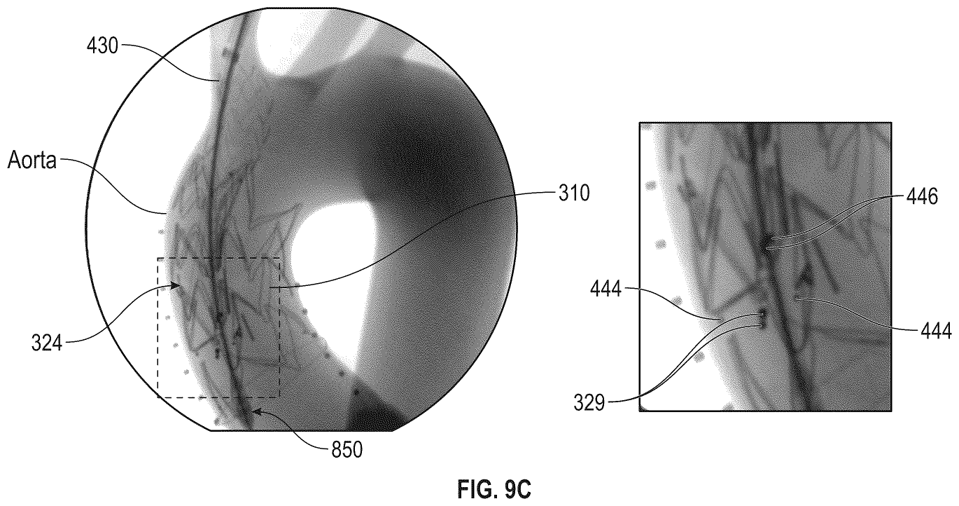

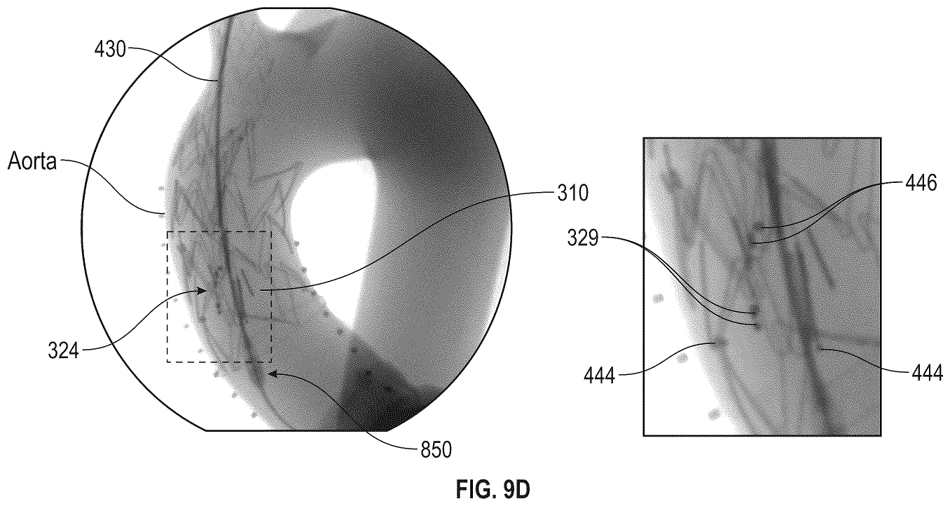

Referring to FIGS. 4B and 4D, the leg member 430 can further include one or more (e.g., two) radiopaque markers 444 to facilitate visualization of the leg member 430 during delivery of the leg member 430 to an aorta and deployment therein. For example, in the illustrated embodiment the leg member 430 includes a pair of the markers 444 positioned on opposing portions of the leading end portion 431 of the leg member 430. The markers 444 can be positioned at (or at least proximate to) corners 445 of the leading end portion 411 defined by/at the connection between the flat portions of the first stents 436a and the curved portions of the first stents 436a. In some embodiments, the leg member 430 can further includes one or more (e.g., two) secondary radiopaque markers 446 aligned with and/or positioned distally of the markers 444. As described in greater detail below with reference to FIGS. 9A-9D, the markers 444, 446 can facilitate rotational and/or longitudinal alignment of the leg member 430 during delivery such that, for example, the D-shape of the coupling portion 440 can be aligned within the secondary lumen 324 of the base member 310 (FIGS. 3A-3D).

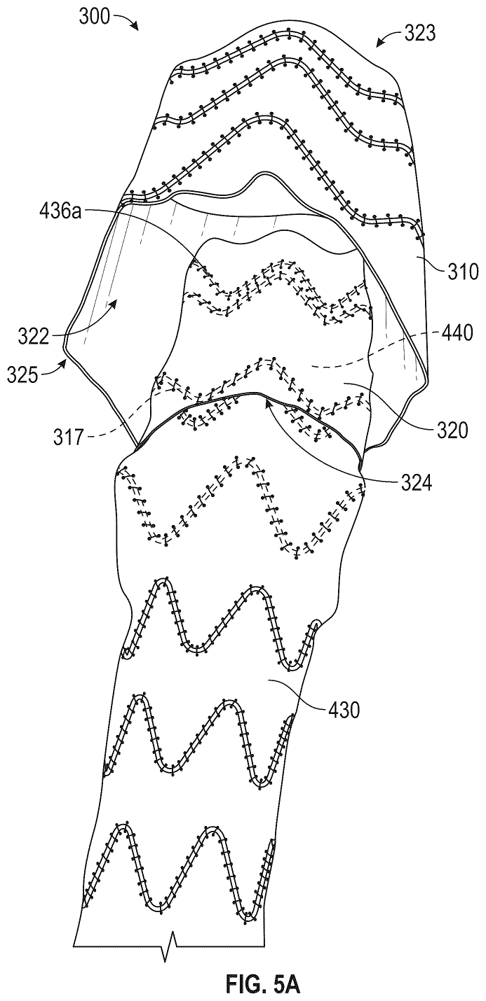

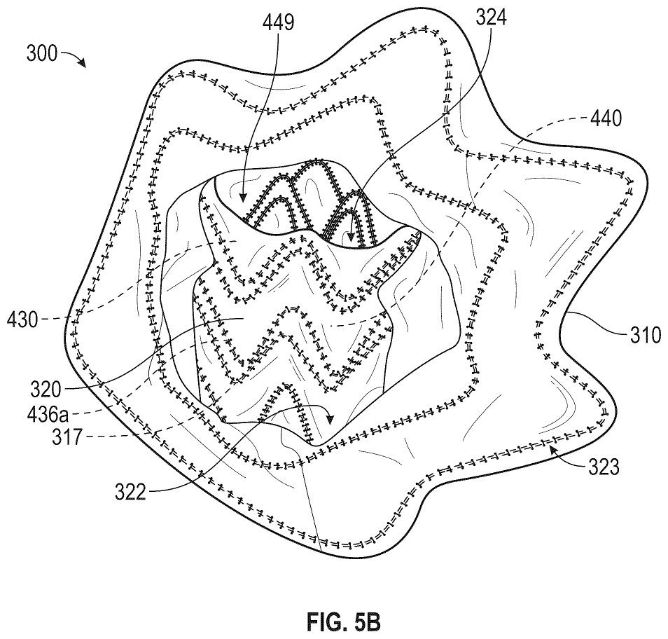

Referring to FIGS. 3A-4D, the leg member 430 and the base member 310 can be separate components that can be modularly coupled together during device deployment in situ. FIG. 5A, for example, is a perspective view of the aortic repair device 300 including the base member 310 of FIGS. 3A-3D coupled to the leg member 430 of FIGS. 4A-4D in accordance with embodiments of the present technology, and FIG. 5B is a perspective view taken from a leading end portion of the aortic repair device 300 of FIG. 5A. Referring to FIGS. 5A and 5B, at least some of the coupling portion 440 of the leg member 430 can be deployed and secured within the secondary lumen 324 of the base member 310. The coupling portion 440 can expand when positioned within the secondary lumen 324 and press against the septum 320 and the inner surface of graft material 318 to form a seal between the graft material 318 and the septum 320 of the base member 310 such that fluid (e.g., blood) entering the leading opening 323 of the base member 310 is routed into and through (i) the primary lumen 322 to the trailing opening 325 (FIG. 5A) of the base member 310 and (ii) the leg lumen 449 (FIG. 5B) to the trailing opening 439 of the leg member 430. In various embodiments, the leg member 430 can be oversized with respect to the cross-sectional dimension of the secondary lumen 324 (i.e., have a larger diameter than that of the secondary lumen 324; e.g., 5% oversizing, less than 5% oversizing, more than 5% oversizing) facilitate the formation a seal between the outer surface of the leg member 430 and the inner surface of the base member 310. Further, oversizing increases the frictional engagement of the leg member 430 with the opposing portions of the base member 310, thereby enhancing fixation between the two members 310, 430 and increasing the force required to separate the two members 310, 430 from each other.

Referring to FIGS. 3A-5B, the leg member 430 can be rotationally and longitudinally aligned within the secondary lumen 324 of the base member 310 (e.g., via visualization of the markers 444 of the leg member 430 and/or the markers 329 of the base member 310) such that, for example, the D-shape of the coupling portion 440 algins with (e.g., matches, mates with) the D-shape of the secondary lumen 324. In some embodiments, the second stents 317 of the base member 310 can have the same or substantially similar height along the longitudinal axis L (FIG. 3A) and/or can have the same or a similar periodicity about the longitudinal axis L as the first stents 436a of the leg member 430. In some embodiments, as shown in FIGS. 5A and 5B, the stents 317, 436a can be aligned in phase with one another when the leg member 430 is coupled to the base member 310. In other embodiments, the stents 317, 436a can be aligned out of phase with another and/or can have different heights, periodicities, etc. In other embodiments, the coupling portion 440 of the leg member 430 can have different shapes to match, for example, a different cross-sectional shape of the secondary lumen 324 of the base member 310. In various embodiments, the interface between the second stents of the leg member 430 and the luminal and internal stents of the base member 310 along the secondary lumen 324 can increase frictional engagement between the two members 310, 430 and increase resistance to separation. The contours of the stents and the orientation of the stents relative to each other can be selected (e.g., out of phase with each other, aligned, differently shaped in each member and/or along different sections of the individual members) to enhance this frictional engagement.

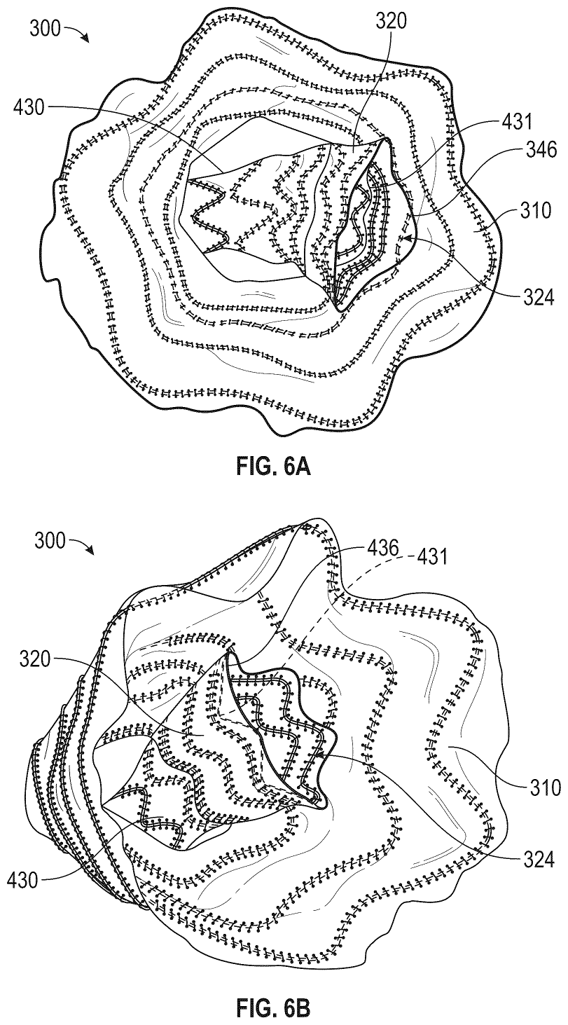

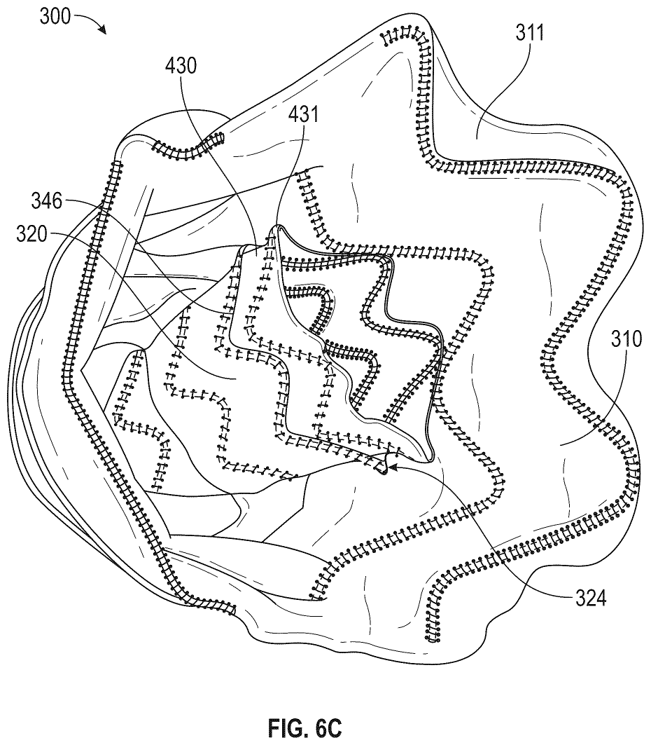

The coupling portion 440 of the leg member 430 can be affixed within the secondary lumen 324 of the base member 310 at any point along its length (e.g., may overlap to varying degrees). Accordingly, an overall length of the aortic repair device 300 can be adjusted by positioning shorter or longer segments of the leg member 430 within the secondary lumen 324 of the base member 310. For example, FIGS. 6A-6C are perspective end on views of the aortic repair device 300 with the leg member 430 inserted at different positions within the secondary lumen 324 of the base member 310 in accordance with embodiments of the present technology. Referring to FIGS. 6A-6C, the septum 320 extends only partially through the base member 310 and terminates at the leading end portion 346 that defines a leading end portion of the secondary lumen 324. In FIG. 6A, the leg member 430 is inserted only partially into the secondary lumen 324 such that the leading end portion 431 (FIGS. 4A-4D) of the leg member 430 is positioned within the secondary lumen 324 away (e.g., in the trailing direction) from the leading end portion 346 of the septum 320. Referring to FIG. 6B, the leg member 340 is inserted farther (e.g., fully) into the secondary lumen 342 such that the leading end portion 431 of the leg member 430 is positioned at and/or adjacent to the leading end portion 346 of the septum 320. Accordingly, in FIG. 6B, the aortic repair device 300 has an overall shorter length than in FIG. 6B because there is more overlap between the base member 310 and the leg member 430. Referring to FIG. 6C, the leg member 430 is inserted farther into the secondary lumen 342 such that the leading end portion 431 of the leg member 430 is positioned outside the secondary lumen 324 past the leading end portion 346 of the septum 320 toward the leading end portion 311 of the base member 310. Accordingly, in FIG. 6C, the aortic repair device 300 has an overall shorter length than in FIG. 6B because there is more overlap between the base member 310 and the leg member 430. In any of the positions illustrated in FIGS. 6A-6C, the leg member 430 can expand within the secondary lumen 324 and be aligned therein to lock the position of the leg member 430 relative to the base member 310.

Referring to FIGS. 3A-6C, in some embodiments the septum 320 can be omitted. In such embodiments, the second stents 317 can define the secondary lumen 324 for receiving the leg member 430.

### III. Selected Embodiments of Methods and Systems for Delivering Aortic Repair Devices

Referring to FIGS. 3A-6C, the aortic repair device 300 can be delivered to a diseased aorta in a modular manner such that the base member 310 and the leg member 430 are deployed sequentially. For example, the base member 310 can be housed in a distal portion of a catheter system with the base member 310 positioned in a delivery state (e.g., a collapsed configuration, compressed state, first state), and the distal portion of the catheter system can access the aorta via any suitable intravascular path-such as an aortic approach, a transfemoral approach, a transcarotid approach, a transsubclavian approach, and so on. The base member 310 can then be deployed from the catheter system at a target site within the aorta, such as adjacent (e.g., downstream from, upstream of) or across an aneurysm or dissection therein. The deployed base member 310 can form a fluid-tight seal against the inner wall of the aorta and thereby route blood through the base member 310. Depending upon the anatomy, indication, and/or desired repair, during the same or a separate, subsequent procedure, the leg member 430 can be intravascularly delivered to the target site via any of the suitable intravascular approaches above, and positioned such that a portion of the leg member 430 is positioned within the secondary lumen 324 of the base member 310 where it can be deployed therein to form a blood impervious seal. The trailing end portion 433 of the leg member 430 can extend from the base member 310 to a desired target site, as within a branch vessel (e.g., the brachiocephalic artery) and deployed to form a seal with the adjacent vessel wall. Upon deployment of the leg member 430, the base member 310 and the leg member 430 can define two flow channels such that blood bypasses the diseased anatomy and exits in non-diseased or healthier portions of the vasculature.

FIGS. 7A-10B more particularly illustrate various aspects of methods and systems for delivering the aortic repair device 300 to, and deploying the aortic repair device 300 within, an aorta. FIGS. 7A-7C are side views (e.g., fluoroscopic images) of various stages of a procedure to implant the base member 310 within a model of an aorta of a patient in accordance with embodiments of the present technology. Referring to FIG. 7A, in a first stage the base member 310 can be constrained within an outer catheter 752 of a delivery system 750 and delivered to the aorta. The delivery system 750 can include some features that are at least generally similar in structure and function, or identical in structure and function, to any of the delivery systems disclosed in U.S. patent application Ser. No. 18/784,740, Filed Jul. 25, 2024, and titled “DELIVERY SYSTEMS FOR AORTIC ARCH REPAIR DEVICES, AND ASSOCIATED DEVICES AND METHODS,” which is incorporated by reference herein in its entirety. In some embodiments, the delivery system 750 is advanced along a transfemoral or transcarotid access path to within the aorta.

Referring to FIG. 7B, in a second stage the outer catheter 752 (FIG. 7A) can be retracted to permit the base member 310 to at least partially expand within the aorta proximate to a diseased portion of the aorta (e.g., a tear, dissection, aneurysm, etc.). In the illustrated embodiment, the delivery system 750 includes an inner catheter 754 and a tip capture mechanism 756 configured to releasably couple the base member 310 to the inner catheter 754. More specifically, the tip capture mechanism 756 can releasably secure a leading one of the stents 316 (identified as leading stent 316a) to the inner catheter 754. The tip capture mechanism 756 can be actuated to release some or all of the leading stent 316a in one or more stages. The tip capture mechanism 756 can be secured to the leading stent 316a as the outer catheter 752 is retracted to provide forward traction on the base member 310 to inhibit or even prevent the base member 310 from moving (e.g., springing distally and/or proximally) during deployment. Further, in the illustrated embodiment, the markers 329 are visible in the fluoroscopic image to allow an operator to visualize the position of the septum 320, the primary lumen 322, and/or the secondary lumen 324 (FIGS. 3A-3D). An operator (e.g., a physician) can rotate the delivery system 750 (e.g., the inner catheter 754) to rotate and align the base member 310 within the aorta. For example, the base member 310 can be rotated such that the first side portion 312 is positioned adjacent to an outer portion of the aorta having the greater curvature and the second side portion 314 positioned adjacent to an inner portion of the aorta having the lesser curvature. In other embodiments, the base member 310 can be rotated and aligned within the aorta while still constrained within the outer catheter 752 (FIG. 7A), and/or with more or less of the base member 310 exposed and deployed from within the outer catheter 752.

Referring to FIG. 7C, in a third stage the tip capture mechanism 756 of the delivery system 750 can be actuated to release the base member 310 within the aorta and allow the base member 310 to fully expand within the aorta into contact with an interior wall of the aorta. Once expanded, the graft material 318 (FIGS. 3A-3D) can seal against the wall of the aorta such that blood flows through the base member 310 past the diseased portion of the aorta.

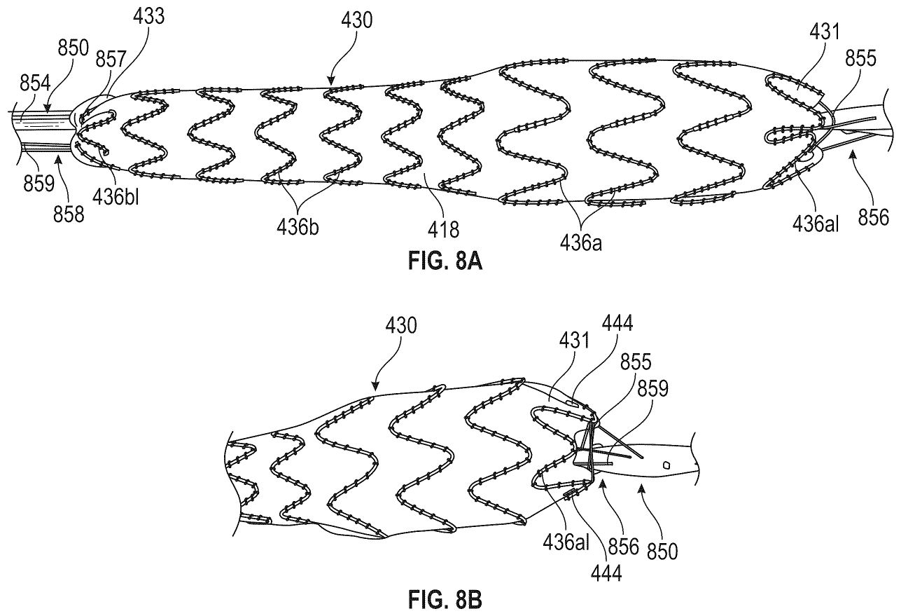

FIG. 8A is a perspective side view of a distal portion of a delivery system 850 secured to the leg member 430 of FIGS. 4A-4D in accordance with embodiments of the present technology. In the illustrated embodiment, the first stents 436a are secured to the outer surface of the graft material 418 (e.g., in an abluminal arrangement) rather than to the inner surface of the graft material 418 (e.g., in an intraluminal arrangement) as shown in FIGS. 4A-4D. The delivery system 850 can include some features that are at least generally similar in structure and function, or identical in structure and function, to any of the delivery systems disclosed in U.S. patent application Ser. No. 18/784,740, Filed Jul. 25, 2024, which is incorporated by reference previously herein.

In the illustrated embodiment, the delivery system 850 includes an inner catheter 854 releasably secured to the leg member 430 via a leading tip capture mechanism 856 and a trailing tip capture mechanism 858. The leading tip capture mechanism 856 can releasably secure the leading end portion 431 of the leg member 430 (e.g., a leading one of the first stents 436a designated 436al) to the inner catheter 854 via one or more first loops 855 (e.g., suture loops). Similarly, the trailing tip capture mechanism 858 can releasably secure the trailing end portion 433 of the leg member 430 (e.g., a trailing one of the second stents 436b designated 436bl) to the inner catheter 854 via one or more second loops 857 (e.g., suture loops). The delivery system 850 further includes one or more release wires 859 that extend through corresponding ones of the first and/or second loops 855, 857 to releasably secure the leading and trailing end portions 431, 433 of the leg member 430 to the inner catheter 854. During deployment, the release wire 859 can be retracted proximally (e.g., to the left of the page in FIG. 8A) to release the first and/or second loops 855, 857 to correspondingly release the leading and trailing end portions 431, 433 of the leg member 430.

FIG. 8B is an enlarged view of the leading end portion 431 of the leg member 430 and the leading tip capture mechanism 856 of the delivery system 850 in accordance with embodiments of the present technology. Referring to FIGS. 8A and 8B, the leading tip capture mechanism 856 can loosely secure the leading first stent 436al to the inner catheter 854 (obscured in FIG. 8B) proximate to the markers 444 and can tightly secure the leading first stent 436al to the inner catheter 854 elsewhere. For example, the leading tip capture mechanism 856 can include multiple ones of the first loops 855 with varying tensions that capture/secure different leading apices of the leading first stent 436al (e.g., with a loose one of the first loops 855 securing the leading apices of the leading first stent 436al proximate the markers 444 and a tighter one of the first loops 855 securing the other leading apices of the leading first stent 436al), as described in detail in U.S. patent application Ser. No. 18/784,740, Filed Jul. 25, 2024, which is incorporated by reference previously herein. In some embodiments, the different ones of the first loops 855 can be released by the same or a different one of the release wires 859 in different stages such that the leading end portion 431 of the leg member 430 is deployed in stages. For example, in some embodiments with a D-shaped end portion, one or more of the apices along a curved or domed region of the leading first stent 436al (e.g., the curved portion of a D-shaped stent) can be captured together with a first wire, while one or more of the apices along a generally flat or bowed region can be captured by a second wire, thereby allowing the curved region to be released as a unit together and the flat region to be released together at a different time from the curved region. In this and other embodiments, the corner apices (e.g., two apices at the intersection of the curved and flat regions forming the D) can be captured by a third wire, or each corner apex can have a dedicated wire (e.g., a third wire for one corner apex and a fourth wire for the other corner apex). In some embodiments, the release wires can be coupled to one or more tab(s) for release from a handle (not shown). This staged release is expected to provide enhanced apex organization and orientation at the leading end. Apex organization during loading can be maintained with a shaped funnel to maintaining grouping of following apices (i.e., non-tip captured apices). That is, the apices from the domed region can be brought together, and the apices from the flat region can be brought together. Accordingly, in some aspects of the present technology the leading tip capture mechanism 856 secures the leading end portion 431 of the leg member 430 to the inner catheter 854 in a partially open state in which the markers 444 are sufficiently spaced apart from another to facilitate visualization thereof for rotationally aligning the leg member 430 during delivery.

FIGS. 9A-9D are side views (e.g., fluoroscopic images) of various stages of a procedure to implant the leg member 430 of FIGS. 4A-4D, 8A, and 8B within a model of an aorta of a patient and to couple the leg member 430 to the base member 310 of FIGS. 3A-3D and 7A-7C in accordance with embodiments of the present technology. Referring to FIGS. 9A-9D, the base member 310 has been previously implanted within the aorta as described in detail above with reference to FIGS. 7A-7C.

Referring to FIG. 9A, in a first stage the leg member 430 can be constrained within an outer catheter 952 of the delivery system 850 and delivered to within the aorta. In some embodiments, the delivery system 850 is advanced along a transcarotid path to within the aorta. In the illustrated embodiment, the outer catheter 952 is inserted at least partially into the secondary lumen 324 of the base member 310 through the trailing opening 325. As best seen in the enlarged view of FIG. 9A, the markers 329 of the base member 310 and the markers 444, 496 of the leg member 430 are visible in the fluoroscopic image. In some embodiments, the markers 444 provide an indication of a longitudinal position of the leading end portion 431 (FIGS. 4A-4D) of the leg member 430 relative to the base member 310 and, more specifically, relative to the leading end portion 346 of the septum 320 (FIGS. 3A-3D) marked by the markers 329. An operator of the delivery system 850 (e.g., a physician) can therefore align the leading end portion 431 of the leg member 430 at a desired position relative to the leading end portion 346 of the septum 320, such as at any of the positions shown in FIGS. 6A-6C. The secondary markers 446 can provide an indication of a rotational orientation of the leg member 430 relative to the base member 310 such that the markers 444 can be referenced for (e.g., solely for) the longitudinal position of the leg member 430. For example, the operator can compare the second markers 446 to the markers 329 on the base member 310 to verify that the leg member 430 is rotationally aligned with the base member 310. In some embodiments, however, the secondary markers 446 are omitted and the markers 444 can be used for longitudinal positioning and rotational alignment. Thus, while FIGS. 9B-9D and the associated descriptions references both the markers 444 and the secondary markers 446 (“markers 444, 446”), those of ordinary skill in the art will appreciate that the secondary markers 446 may be omitted and the markers 444 may be used in their place.

Referring to FIG. 9B, in a second stage the outer catheter 952 of the delivery system 850 can be partially retracted to deploy (e.g., unsheathe) a leading portion of the leg member 430, such as some or all of the coupling portion 440 (FIGS. 4A-4C). Referring to FIGS. 8A, 8B, and 9B, the leading tip capture mechanism 856 can secure the leading first stent 436al to the delivery system 850 while allowing the leading end portion 431 to partially expand to permit visualization of the markers 444, 446 (best seen in the enlarged view of FIG. 9B). At this second stage, the delivery system 850 (e.g., the inner catheter 854) can be rotated to align the D-shape of the coupling portion 440 (FIGS. 4A-4C) within the secondary lumen 324 of the base member 310 based on the positioning of the markers 444, 446.

Referring to FIG. 9C, in a third stage the outer catheter 952 (FIGS. 9A and 9B) of the delivery system 850 can be further retracted to fully deploy (e.g., unsheathe) the leg member 430. In some embodiments, the leg member 430 extends from the base member 310 and into a branch vessel (e.g., the brachiocephalic artery) such that the trailing end portion 433 and the trailing opening 439 (FIGS. 4A and 4B) are positioned within the branch vessel.

Referring to FIG. 9D, in a fourth stage, the leading tip capture mechanism 856 (FIGS. 8A and 8B; and/or the trailing tip capture mechanism 858) can be actuated to release the leg member 430 from the delivery system 850. For example, referring to FIGS. 8A, 8B, and 9D, one or more of the release wires 859 can be retracted to release the first and second loops 855, 857 to release the leg member 430 from the inner catheter 854 in one or more stages. After release from the delivery system 850, the leg member 430 can fully expand such that the graft material 438 (FIGS. 4A-4D) seals against the base member 310 within the secondary lumen 324.

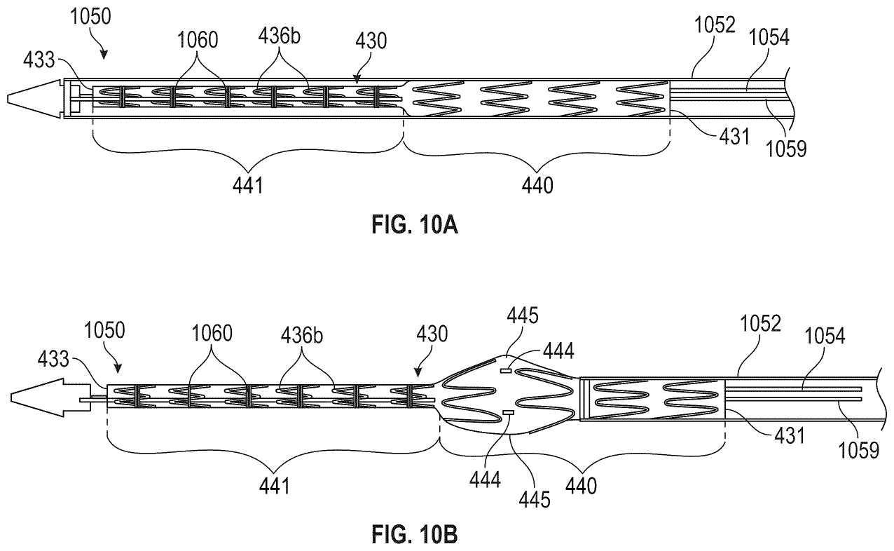

FIGS. 10A and 10B are side views of different stages of deploying the leg member 430 of FIGS. 4A-4D with a delivery system 1050 in accordance with additional embodiments of the present technology. The delivery system 1050 can include some features that are at least generally similar in structure and function, or identical in structure and function, to the delivery systems 750 and 850 described in detail above with reference to FIGS. 7A-9D and/or any of the delivery systems disclosed in U.S. patent application Ser. No. 18/784,740, Filed Jul. 25, 2024, which is incorporated by reference previously herein. In particular, the delivery system 1050 can include features similar in structure and function to the delivery system described in detail with reference to FIGS. 38A-39C of U.S. patent application Ser. No. 18/784,740, Filed Jul. 25, 2024.

Referring to FIGS. 10A and 10B, the delivery system 1050 includes an outer catheter 1052, an inner catheter 1054, and a release wire 1059. In the illustrated embodiment, the leg member 430 further includes one or more constraining fibers 1060 (e.g., threads, metal wireless, sutures) secured to, for example, one or more of the stents second stents 436b via suturing, knotting, and/or the like. The constraining fibers 1060 can each include a pair of loops that extend (e.g., wrap) around the leg portion 441 and that are secured together via the release wire 1059. The release wire 1059 can be integrated to include other wire functions (e.g., tip capture release), or can be a separate wire dedicated to hold, engage, and subsequently release the constraining fibers 1060. In the illustrated embodiment, the leg member 430 is oriented opposite to the configuration shown in FIGS. 8A and 8B—that is, with the end portion 433 of the leg portion 441 defining a leading end portion (“leading end portion 433”) and the end portion 431 of the coupling portion 440 defining a trailing end portion (“trailing end portion 431”).

Referring to FIG. 10A, in a first stage the leg member 430 has been compressed into the outer catheter 1052 of the delivery system 1050. Referring to FIG. 10B, in a second stage the outer catheter 1052 has been retracted (e.g., proximally) to partially deploy the leg member 430 from the outer catheter 1052. In the illustrated embodiment, the leg portion 441 is deployed from the outer catheter 1052 and the coupling portion 440 is partially deployed from the outer catheter 1052. The engagement of the constraining fibers 1060 with the release wire 1059 maintains the leg portion 441 in a compressed state even after deployment from the outer catheter 1052. The coupling portion 440 can at least partially expand when deployed from the outer catheter 1052 to, for example, permit the markers 444 to move apart from one another to facilitate visualization of the corners 445 of the coupling portion 440 and thus the rotational orientation of the leg member 430. In some aspects of the present technology, by maintaining the leg portion 441 in the compressed state, the leg member 430 can be rotated to a desired orientation/alignment (e.g., within the secondary lumen 324 of the base member 310 of FIGS. 3A-3D). After rotational alignment, the leg portion 441 can be selectively expanded within the aorta and/or a branch vessel via retraction of the release wire 1059 past a subset (e.g., one or more) of the constraining fibers 1060.

### IV. Select Embodiments of Aortic Treatment via Modular Aortic Repair Devices, Such as Full Aortic Arch Treatment

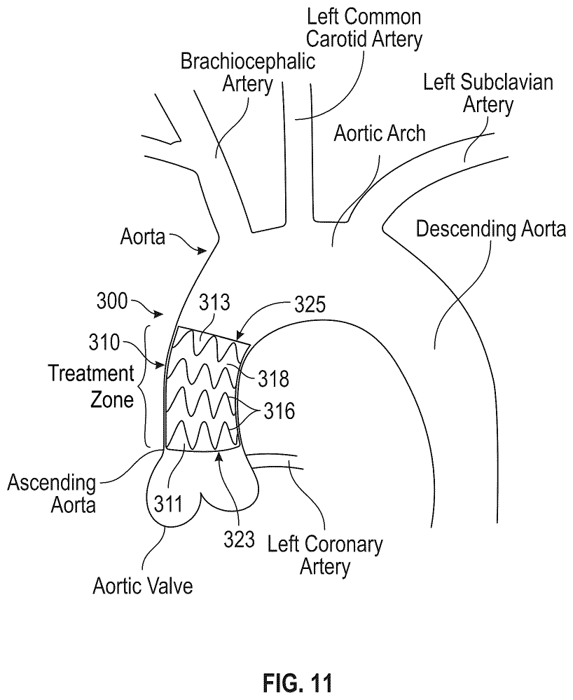

FIG. 11 is a side view of the base member 310 of the aortic repair device 300 of FIGS. 3A-3D implanted within an aorta after implantation via the delivery system 750 of FIGS. 7A-7C in accordance with embodiments of the present technology. In some embodiments, the aorta can include an aneurysm, dissection, and/or other diseased portion as described in detail above with reference to FIGS. 1A-2B. In the illustrated embodiment, the base member 310 is implanted within the proximal aorta (e.g., the ascending aorta and/or the aortic arch) with the leading end portion 311 positioned proximate to the aortic valve. The stents 316 can expand the graft material 318 into contact with the inner wall of the aorta to provide a seal between the aorta and the base member 310. More particularly, referring to FIGS. 3A-3D and 11, the base member 310 can sealingly contact the inner wall of the proximal aorta to define a treatment zone and such that all or substantially all blood flow through the aorta enters the leading opening 323 and flows through either the primary lumen 322 or the secondary lumen 324 to the trailing opening 325 at the trailing end portion 313. In some aspects of the present technology, the base member 310 can be positioned against/adjacent to the diseased portion of the aorta, such as the origin of a dissection and/or aneurysm, to block blood flow into the diseased portion by diverting the blood flow through the base member 310 and past the diseased portion.

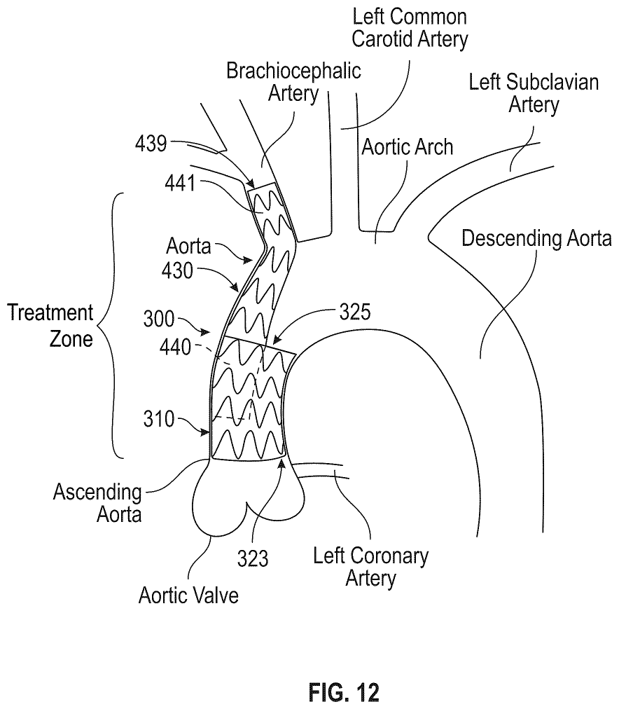

In some embodiments, the treatment zone provided by the base member 310 may not be sufficiently large to treat the diseased portion of the aorta. Accordingly, in some embodiments the leg member 430 (FIGS. 4A-4D) can be coupled to the base member 310 to extend the treatment zone during the same or a separate procedure used to implant the base member 310 as described in detail above with reference to FIGS. 8A-9D. FIG. 12, for example, is a side view of the aortic repair device 300 with the leg member 430 coupled to the base member 310 in accordance with embodiments of the present technology. In the illustrated embodiment, the coupling portion 440 of the leg member 430 is positioned within the secondary lumen 324 (FIGS. 3C and 3D) and sealingly engages the base member 310 within the secondary lumen 324. The leg portion 441 of the leg member 430 extends at least partially into a branch artery, such as the brachiocephalic artery. Accordingly, referring to FIGS. 3A-4D and 12, the base member 310 can direct the blood flow (i) through the primary lumen 322 and out of the trailing opening 325 into the aorta to perfuse the aorta and (ii) into the leg lumen 449 of the leg member 430 positioned within the secondary lumen 324. The leg lumen 449 can direct the blood flow received therein out of the trailing opening 439 into the brachiocephalic artery to perfuse the brachiocephalic artery. In some aspects of the present technology, the leg portion 441 can extend the treatment zone within the aorta.

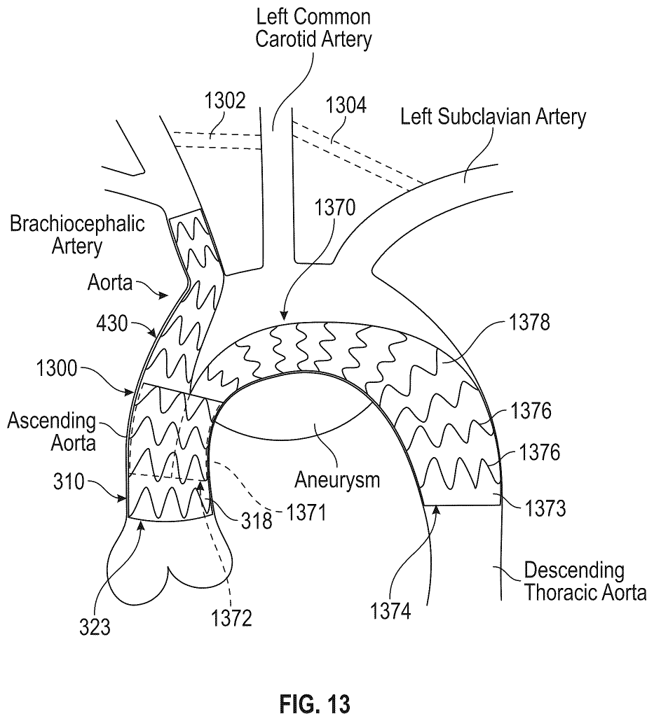

In other embodiments, additional components/devices/members can be fluidly coupled to the base member 310 to provide for further treatment of the aorta. FIG. 13, for example, is a side view of an aortic repair device 1300 implanted within an aorta in accordance with additional embodiments of the present technology. In the illustrated embodiment, the aortic repair device 1300 includes the base member 310 and the leg member 430 described in detail above, and further includes a separate spanning member 1370 coupled to (e.g., attached to, docked to) the base member 310. The spanning member 1370 can have a tubular shape defining a lumen and can include features generally similar to those of the base member 310 and/or the leg member 430. For example, in some embodiments the spanning member 1370 is an expandable stent graft comprising one or more struts or stents 1376 and a graft material 1378 coupled to the stents 1376.

In the illustrated embodiment, the spanning member 1370 includes a leading end portion 1371 (e.g., a first end portion, a proximal end portion) defining a leading terminus of the spanning member 1370 and a trailing end portion 1373 (e.g., a second end portion, a distal end portion) defining a trailing terminus of the spanning member 1370. The spanning member 1370 can be generally hollow and define one or more lumens (e.g., flow conduits) therethrough. Accordingly, in the illustrated embodiment the spanning member 1370 includes a leading opening (e.g., a fluid opening, a first spanning fluid opening, a leading spanning fluid opening, and/or the like) 1372 and a trailing opening 1374 (e.g., a fluid opening, a second spanning fluid opening, a trailing spanning fluid opening, and/or the like).

Referring to FIGS. 3A-3D and 13, the leading end portion 1371 of the spanning member 1370 can be at least partially positioned within the base member 310 within the primary lumen 322 and can sealingly engage the base member 310 within the primary lumen 322 to define a continuous blood flow path from the leading opening 323 of the base member 310, into the leading opening 1372 of the spanning member 1370, and through the lumen of the spanning member 1370 to the trailing opening 1374. That is, the graft material 1378 of the spanning member 1370 can sealingly engage the septum 320 and the graft material 318 of the base member 310 within the primary lumen 322 such that blood flow is routed through the base member 310 to the lumen of the spanning member 1370. In some embodiments, at least a portion of the spanning member 1370 (e.g., the trailing end portion 1373) sealingly engages the aorta within the aortic arch and/or the descending thoracic aorta. Accordingly, the aortic repair device 1300 can direct blood flow through the leg member 430 to the brachiocephalic artery and through the spanning member 1370 to the descending thoracic aorta. Accordingly, in some aspects of the present technology the aortic repair device 1300 can divert blood flow past a diseased portion of the aorta, such an aneurysm in the aortic arch shown in FIG. 13. The spanning member 1370 can be delivered to the aorta in a collapsed configuration within a delivery system in the same or a separate procedure as the base member 310 and/or the leg member 430.

Often a patient may initially only need treatment within the ascending aorta and/or the aortic arch to treat an initial dissection or aneurysm but, at a later time (e.g., months or years later), may require additional treatment of the aortic arch and/or the descending thoracic aorta as the diseased state progresses. Accordingly, the base member 310 (and possibly the leg member 430) can be implanted during an initial procedure and the spanning member 1370 can be implanted during a later procedure and modularly coupled to the base member 310 to provide further treatment of the aortic arch and/or the descending thoracic aorta (e.g., by bypassing blood flow past the aneurysm shown in FIG. 13).

Referring to FIG. 13, the spanning member 1370 bypasses the left common carotid artery and the left subclavian artery and thus may partially or fully occlude those vessels. In some embodiments, a bypass 1302 between the perfused right common carotid artery and/or a bypass 1304 between the left common carotid artery and the left subclavian artery (and/or between the brachiocephalic artery and the left subclavian artery) can be surgically created to perfuse those vessels. In other embodiments, the aortic repair device 1300 can include additional implantable devices (e.g., stent grafts) coupled to the spanning member 1370 and/or the base member 310 that are configured (e.g., sized, shaped, positioned) to perfuse different branch vessels.

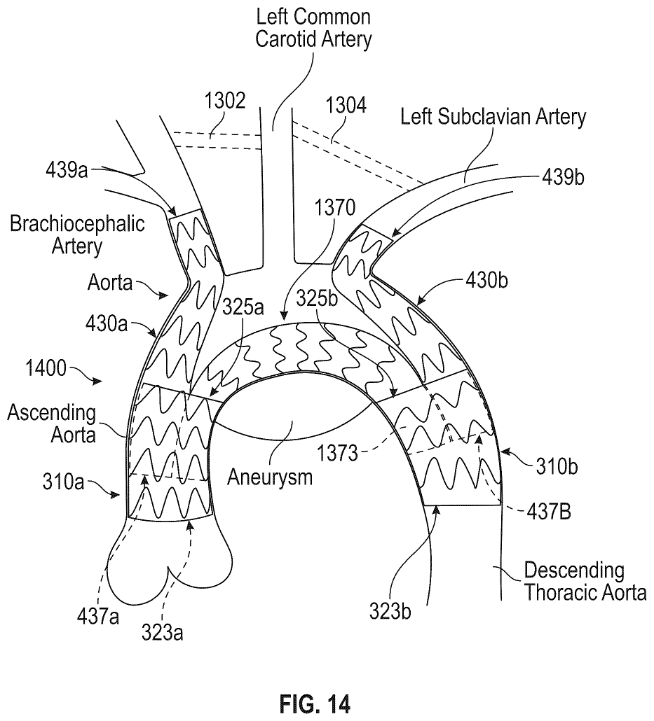

FIG. 14 is a side view of an aortic repair device 1400 implanted within an aorta in accordance with additional embodiments of the present technology. In the illustrated embodiment, the aortic repair device 1400 includes the base member 310 (identified as first base member 310a), the leg member 430 (identified as first leg member 430a), and the spanning member 1370 described in detail above. In the illustrated embodiment, the aortic repair device 1400 further includes a second base member 310b implanted at least partially within the descending thoracic aorta and a second leg member 430b coupled to the second base member 310b. The second base member 310b and the second leg member 430b can be identical or substantially identical to the first base member 310a and the first leg member 430a, respectively. For example, in the illustrated embodiment (i) the first base member 310a defines a leading opening 323a and a trailing opening 325a and (ii) the second base member 310b defines a leading opening 325b and a trailing opening 323b. Similarly, (i) the first leg member 430a defines a leading opening 437a and a trailing opening 439a and (ii) the second leg member 430b defines a leading opening 437b and a trailing opening 439b. While the first and second base members 310a-b can be identical or substantially identical, because the orientation of the first and second base members 310a-b is reversed in FIG. 14, the leading opening 323a and the trailing opening 325a of the first base member 310a are equivalent in structure to the trailing opening 323b and the leading opening 325b, respectively, of the second base member 310b.

The spanning member 1370 spans from the base member 310a and is coupled to the second base member 310b. Specifically, with reference to FIGS. 3A-3D and 14 together, the trailing end portion 1373 of the spanning member 1370 can be positioned within the second base member 310b within the primary lumen 322 and can sealingly engage the second base member 310b within the primary lumen 322 to define a continuous blood flow path from the spanning member 1370 through the primary lumen 322 of the second base member 310b, and out of the trailing (e.g., distal) opening 323b. The second leg member 430b can extend into a second branch artery (e.g., the left subclavian artery, left carotid) for perfusing the second branch artery. For example, the second base member 310b can receive retrograde blood flow through the trailing opening 323b for perfusing the second branch artery. Alternatively or additionally, the secondary lumen 375 of the second base member 310b can be perfused via the primary lumen 373 where the septum 320 extends only partially from the leading end portion 311 toward the trailing end portion 313. That is, blood flow from the spanning member 1370 can flow into the primary lumen 322 and around the septum 320 into the leading opening 437b of the second leg member 430b where the septum 320 terminates within the second base member 310b. In some aspects of the present technology, the aortic repair device 1400 can provide for a full arch treatment in which (i) the first leg member 430a directs blood flow to a first branch artery (e.g., the brachiocephalic artery), (ii) the second leg member 430b directs blood flow to a second branch artery (e.g., the left subclavian artery), (iii) a third branch artery (e.g., the left common carotid artery) is perfused via the bypass 1302 and/or 1304, and (iv) the primary lumens 322 of the first and second base members 310a-b and the spanning member 1370 collectively direct blood flow to the descending thoracic aorta.

In some embodiments, the second base member 310b and the second leg member 430b can be omitted and the spanning member 1370 can be coupled to another aortic repair device, such as any of the aortic repair devices described in U.S. patent application Ser. No. 18/179,254, Filed Mar. 6, 2023, and titled “DEVICES FOR AORTIC REPAIR, AND ASSOCIATED SYSTEMS AND METHODS,” which is incorporated by reference previously herein. For example, the second base member 310b and the second leg member 430b can be integrated into a single, integral stent graft.

### V. Additional Examples

The following examples are illustrative of several embodiments of the present technology:
1. An aortic arch repair device, comprising:
a base member having a first end portion and a second end portion, the base member comprising—
   a plurality of first stents,
   a plurality of second stents, and
   a cover coupled to the plurality of first stents and the plurality of second stents, the cover defining a fluid conduit through the base member,
   wherein—
      the first end portion of the base member defines a first fluid opening, and
      at least a portion of the cover is positioned between the plurality of first stents and the plurality of second stents;
   a septum coupled to the plurality of second stents and extending through the base member from the second end portion of the base member toward the first end portion of the base member, wherein—
      the septum divides the fluid conduit into a primary lumen having a first cross-sectional area and a secondary lumen having a second cross-sectional area less than the first cross-sectional area, and
      the second end portion of the base member defines a second fluid opening for the primary lumen and a third fluid opening for the secondary lumen; and
   a leg member defining a leg lumen configured to be fluidly coupled to the secondary lumen, the leg member comprising—
      a coupling portion configured to be received within the secondary lumen between the septum and the cover, and
      a leg portion coupled to the coupling portion and configured to extend through the third fluid opening and outwardly from the second end portion of the base member, the leg portion having a leg end portion defining a fourth fluid opening for the secondary lumen.
2. The aortic arch repair device of example 1 wherein the plurality of first stents comprise a plurality of abluminal stents and wherein the plurality of second stents comprise a plurality of intraluminal stents.
3. The aortic arch repair device of example 1 wherein:
the cover comprises a graft material having an outer surface and an inner surface;
the plurality of first stents are coupled to the outer surface and extend circumferentially about the outer surface, and
the plurality of second stents are coupled to the inner surface and the septum and extend circumferentially about the secondary lumen.
4. The aortic arch repair device of any one of the preceding examples wherein at least one of the plurality of first stents at least partially defines the first fluid opening and wherein at least one of the plurality of second stents at least partially defines the third fluid opening.
5. The aortic arch repair device of any one of the preceding examples wherein the leg member comprises:
a leg member cover having an outer surface and an inner surface,
a plurality of third stents positioned within the coupling portion, wherein individual ones of the plurality of third stents are coupled to the inner surface or the outer surface of the leg member cover, and
a plurality of fourth stents positioned within the leg portion, wherein individual ones of the plurality of fourth stents are coupled to the inner surface or the outer surface of the leg member cover.
6. The aortic arch repair device of example 5 wherein one or more of the plurality of third stents are configured to be expanded when the coupling portion is received within the secondary lumen.
7. The aortic arch repair device of example 5 wherein:
the plurality of second stents have a first cross-sectional shape comprising a first flat portion and a first curved portion,
the plurality of third stents have a second cross-sectional shape comprising a second flat portion and a second curved portion,
the second flat portion is configured to sealingly engage the first flat portion, and
the second curved portion is configured to sealingly engage the first curved portion.
8. The aortic arch repair device of example 7, further comprising:
a pair of first markers, each of the pair of first markers positioned at a respective first intersection of the first flat portion and the first curved portion; and
a pair of second markers, each of the pair of second markers positioned at a respective second intersection of the second flat portion and the second curved portion,
wherein the pair of first markers are configured to be aligned with the pair of second markers to rotationally align the base member and the leg member.
9. The aortic arch repair device of example 8 wherein the pair of first markers are a pair of first radiopaque markers and wherein the pair of second markers are a pair of second radiopaque markers.
10. The aortic arch repair device of example 7, further comprising:
a first radiopaque marker at a proximal end of the leg member; and
a second radiopaque marker positioned along the septum of the base member.
11. The aortic arch repair device of any one of the preceding examples wherein none of the plurality of first stents are attached to or extend along the septum.
12. The aortic arch repair device of any one of the preceding examples wherein the coupling portion of the leg member has a first cross-sectional shape and wherein the leg portion of the leg member has a second cross-sectional shape different than the first cross-sectional shape.
13. The aortic arch repair device of example 12 wherein the first cross-sectional shape is D-shaped and the second cross-sectional shape is circular.
14. The aortic arch repair device of any one of the preceding examples wherein:
the coupling portion of the leg member comprises a substantially D-shaped cross-section;
the leg portion of the leg member comprises a circular cross-sectional shape,
and the leg member comprises transition section comprises stents that change from the substantially D-shaped cross-section to the circular cross-section shape.
15. The aortic arch repair device of example 14 wherein the coupling portion has a large cross-sectional dimension than the leg portion.
16. The aortic arch repair device of any one of the preceding examples wherein:
at least one of the plurality of first stents each have a keyhole configuration in which linear first segments extend between second segments at apex portions of the individual first stent, the second segments forming nonzero angles relative to adjacent first segments.
17. An aortic arch repair device, comprising:
a base member having a first end portion and a second end portion, the base member comprising a plurality of stents and a cover coupled to the plurality of stents to define a fluid conduit through the base member, wherein the first end portion of the base member defines a first fluid opening;
a septum coupled to the cover and extending through the base member from the second end portion of the base member toward the first end portion of the base member, wherein—
   the septum divides the fluid conduit into a primary lumen having a first cross-sectional area and a secondary lumen having a second cross-sectional area less than the first cross-sectional area,
   the secondary lumen comprises a generally flat portion adjacent the septum and a curved portion adjacent the cover, the generally flat portion being less curved than the curved portion, and
   the second end portion of the base member defines a second fluid opening for the primary lumen and a third fluid opening for the secondary lumen; and
a leg member defining a leg lumen configured to be fluidly coupled to the secondary lumen, the leg member comprising-—
   a coupling portion configured to be received within the secondary lumen, the coupling portion having—
      a curved surface configured to be aligned with and sealingly engage the curved portion of the secondary lumen, and
      a generally flat surface configured to be aligned with and sealingly engage the generally flat portion of the secondary lumen, the generally flat surface being less curved than the curved surface; and
      a leg portion coupled to the coupling portion and configured to extend through the third fluid opening and outwardly from the second end portion of the base member, the leg portion having a leg end portion defining a fourth fluid opening for the secondary lumen.
18. The aortic arch repair device of example 17 wherein the cover comprises a graft material having an outer surface and an inner surface, and wherein the plurality of stents comprise a plurality of first stents coupled to the outer surface of the graft material and a plurality of second stents coupled to the inner surface of the graft material.
19. The aortic arch repair device of example 18 wherein the plurality of first stents each have a first cross-sectional shape and wherein the plurality of second stents have a second cross-sectional shape different than the first cross-sectional shape.
20. The aortic arch repair device of example 18 wherein the plurality of second stents at least partially define the secondary lumen and individual ones of the plurality of second stents comprise:
a curved section defining the curved portion of the secondary lumen, and
a generally flat section defining the generally flat portion of the secondary lumen, the generally flat section being less curved than the curved section.
21. The aortic arch repair device of any one of the preceding examples wherein the coupling portion is configured to be expanded when positioned within the secondary lumen, and wherein the expansion of the coupling portion creates the sealing engagement between the generally flat surface and the generally flat portion and the curved surface and the curved portion.
22. The aortic arch repair device of any one of the preceding examples wherein the base member extends along a first axis and the leg member extends along a second axis non-parallel to the first axis.
23 The aortic arch repair device of any one of the preceding examples wherein the base member is configured to be positioned in an ascending portion of a thoracic aorta of a patient such that the first fluid opening receives blood flow from the ascending portion of the thoracic aorta and the second fluid opening discharges the blood flow into the thoracic aorta, and wherein at least a portion of the leg member is configured to be positioned within a branch vessel branching from the thoracic aorta.
24. The aortic arch repair device of any one of the preceding examples wherein the base member is configured to be positioned in a descending portion of a thoracic aorta of a patient such that the second fluid opening receives blood flow from the thoracic aorta and the first fluid opening discharges the blood flow into the descending portion of the thoracic aorta, and wherein at least a portion of the leg member is configured to be positioned within a branch vessel branching from the thoracic aorta.
25. A method of repairing a thoracic aorta, the method comprising:
intravascularly delivering a base member of an aortic repair device to a target site in the thoracic aorta;
positioning the base member such that an exterior surface of the base member sealingly contacts a wall of the thoracic aorta, a first fluid opening of the base member is positioned to receive blood flow from the thoracic aorta, and a second fluid opening of the base member is positioned to discharge a first portion of the blood flow into the thoracic aorta,
   wherein the base member comprises a septum extending at least partially between the first fluid opening and the second fluid opening of the base member, the septum dividing the base member into a primary lumen and a secondary lumen, and
   wherein the first fluid opening of the base member is fluidly coupled to the primary lumen to receive the first portion of the blood flow therethrough; and
coupling a leg member of the aortic repair device to the base member such that a third fluid opening of the leg member is positioned to receive a second portion of the blood flow from the secondary lumen and discharge the second portion of the blood flow into a branch vessel branching from the thoracic aorta via a third fluid opening of the leg member, wherein the third fluid opening is fluidly coupled to the secondary lumen to receive the second portion of the blood flow therethrough.
26. The method of example 25 wherein coupling the leg member to the base member comprises:
positioning a leg portion of the leg member within the branch vessel;
positioning a coupling portion of the leg member within the secondary lumen; and
causing the coupling portion to expand and sealing engage the secondary lumen.
27. The method of any one of the preceding examples wherein coupling the leg member to the base member comprises advancing a coupling portion of the leg member further into the secondary lumen to decrease a length of a leg portion of the leg member that extends beyond the base member.
28. The method of example 25 wherein coupling the leg member to the base member comprises withdrawing a coupling portion of the leg member from within the secondary lumen to increase a length of a leg portion of the leg member that extends beyond the base member.
29 The method of any one of the preceding examples wherein coupling the leg member to the base member comprises aligning one or more first markers on the leg member with one or more second markers on the base member.
30. The method of example 29 wherein aligning the one or more first markers with the one or more second markers includes rotating the leg member relative to the base member to rotationally align the one or more first markers with the one or more second markers.
31. The method of any one of the preceding examples wherein positioning the base member comprises rotating the base member to position—
a first side portion of the base member adjacent an outer portion of the thoracic aorta having a greater curvature, and
a second side portion of the base member adjacent an inner portion of the thoracic aorta having a lesser curvature, the second side portion opposite the first side portion.
32. The method of any one of the preceding examples wherein rotating the base member further comprises:
positioning the primary lumen adjacent to an outer portion of the thoracic aorta having a greater curvature; and
positioning the secondary lumen adjacent to an inner portion of the thoracic aorta having a lesser curvature.
33 The method of any one of the preceding examples wherein the branch vessel is a brachiocephalic artery.
34 The method of any one of the preceding examples wherein the branch vessel is a left subclavian artery.
35. The method of any one of the preceding examples wherein the branch vessel is a left common carotid artery.
36. The method of any one of the preceding examples wherein positioning the base member of the aortic repair device includes positioning the base member adjacent to an aortic aneurysm.
37 The method of any one of the preceding examples wherein positioning the base member of the aortic repair device includes positioning the base member adjacent to an aortic dissection.
38. A medical device, comprising:
a base member having a first end portion and a second end portion, the base member comprising—
   a plurality of first stents,
   a plurality of second stents, and
   a cover coupled to the plurality of first stents and the plurality of second stents, the cover defining a fluid conduit through the base member,
   wherein
      the first end portion of the base member defines a first fluid opening, and
      at least a portion of the cover is positioned between the plurality of first stents and the plurality of second stents; and
a septum coupled to the plurality of second stents and extending through the base member from the second end portion of the base member toward the first end portion of the base member, wherein—
   the septum divides the fluid conduit into a primary lumen having a first cross-sectional area and a secondary lumen having a second cross-sectional area less than the first cross-sectional area, and
   the second end portion of the base member defines a second fluid opening for the primary lumen and a third fluid opening for the secondary lumen,
wherein the secondary lumen is configured to receive at least a portion of a leg member and be fluidly coupled to a leg lumen of the leg member, the leg member comprising-—
   a coupling portion configured to be received within the secondary lumen between the septum and the cover, and
   a leg portion coupled to the coupling portion and configured to extend through the third fluid opening and outwardly from the second end portion of the base member, the leg portion having a leg end portion defining a fourth fluid opening for the secondary lumen.
39 The medical device of example 38 wherein the plurality of first stents comprise a plurality of abluminal stents and wherein the plurality of second stents comprise a plurality of intraluminal stents.
40. The medical device of any one of the preceding examples wherein:
the cover comprises a graft material having an outer surface and an inner surface;
the plurality of first stents are coupled to the outer surface and extend circumferentially about the outer surface, and
the plurality of second stents are coupled to the inner surface and the septum and extend circumferentially about the secondary lumen.
41 The medical device of any one of the preceding examples wherein at least one of the plurality of first stents at least partially defines the first fluid opening and wherein at least one of the plurality of second stents at least partially defines the third fluid opening.
42. The medical device of any one of the preceding examples, further comprising the leg member, wherein the leg member comprises:
a leg member cover having an outer surface and an inner surface,
a plurality of third stents positioned within the coupling portion, wherein individual ones of the plurality of third stents are coupled to the inner surface or the outer surface of the leg member cover, and
a plurality of fourth stents positioned within the leg portion, wherein individual ones of the plurality of fourth stents are coupled to the inner surface or the outer surface of the leg member cover.
43 The medical device of example 42 wherein one or more of the plurality of third stents are configured to be expanded when the coupling portion is received within the secondary lumen.
44. The medical device of example 42 wherein:
the plurality of second stents have a first cross-sectional shape comprising a first flat portion and a first curved portion,
the plurality of third stents have a second cross-sectional shape comprising a second flat portion and a second curved portion,
the second flat portion is configured to sealingly engage the first flat portion, and
the second curved portion is configured to sealingly engage the first curved portion.
45 The medical device of any one of the preceding examples wherein none of the plurality of first stents are attached to or extend along the septum.
46. The medical device of any one of the preceding examples wherein the coupling portion of the leg member has a first cross-sectional shape and wherein the leg portion of the leg member has a second cross-sectional shape different than the first cross-sectional shape.
47. The medical device of example 46 wherein the first cross-sectional shape is D-shaped and the second cross-sectional shape is circular.
48. The medical device of any one of the preceding examples wherein the medical device is an aortic arch repair device.
49. An aortic arch repair device, comprising:
a base member having a first end portion and a second end portion, the base member comprising a plurality of stents and a cover coupled to the plurality of stents to define a fluid conduit through the base member, wherein the first end portion of the base member defines a first fluid opening; and
a septum coupled to the cover and extending through the base member from the second end portion of the base member toward the first end portion of the base member,
wherein—
   the septum divides the fluid conduit into a primary lumen having a first cross-sectional area and a secondary lumen having a second cross-sectional area less than the first cross-sectional area,
   the secondary lumen comprises a generally flat portion adjacent the septum and a curved portion adjacent the cover, the generally flat portion being less curved than the curved portion, and
   the second end portion of the base member defines a second fluid opening for the primary lumen and a third fluid opening for the secondary lumen.
50 The aortic arch repair device of example 49 wherein the cover comprises a graft material having an outer surface and an inner surface, and wherein the plurality of stents comprise a plurality of first stents coupled to the outer surface of the graft material and a plurality of second stents coupled to the inner surface of the graft material.
51. The aortic arch repair device of example 50 wherein the plurality of first stents have a first cross-sectional shape and wherein the plurality of second stents have a second cross-sectional shape different than the first cross-sectional shape.
52. The aortic arch repair device of example 50 wherein the plurality of second stents at least partially define the secondary lumen and individual ones of the plurality of second stents comprise:
a curved section defining the curved portion of the secondary lumen, and
a generally flat section defining the generally flat portion of the secondary lumen, the generally flat section being less curved than the curved section.
53. The aortic arch repair device of any one of the preceding examples, further comprising:
a leg member having a coupling portion configured to be received within the secondary lumen and a leg lumen configured to be fluidly coupled to the secondary lumen, wherein the coupling portion comprises a curved surface configured to be aligned with and sealingly engage the curved portion of the secondary lumen and a generally flat surface configured to be aligned with and sealingly engage the generally flat portion of the secondary lumen, the generally flat surface being less curved than the curved surface.
54. The aortic arch repair device of example 53 wherein the leg member comprises a leg portion coupled to the coupling portion and configured to extend through the third fluid opening and outwardly from the second end portion of the base member, the leg portion having a leg end portion defining a fourth fluid opening for the secondary lumen.
55 The aortic arch repair device of example 53 wherein the coupling portion is configured to be expanded when positioned within the secondary lumen, and wherein the expansion of the coupling portion creates the sealing engagement between the generally flat surface and the generally flat portion and the curved surface and the curved portion.
56. The aortic arch repair device of example 53 wherein the base member extends along a first axis and the leg member extends along a second axis non-parallel to the first axis.
57. The aortic arch repair device of example 54 wherein the base member is configured to be positioned in an ascending portion of a thoracic aorta of a patient such that the first fluid opening receives blood flow from the ascending portion of the thoracic aorta and the second fluid opening discharges the blood flow into the thoracic aorta, and wherein at least a portion of the leg member is configured to be positioned within a branch vessel branching from the thoracic aorta.
58. The aortic arch repair device of example 54 wherein the base member is configured to be positioned in a descending portion of a thoracic aorta of a patient such that the second fluid opening receives blood flow from the thoracic aorta and the first fluid opening discharges the blood flow into the descending portion of the thoracic aorta, and wherein at least a portion of the leg member is configured to be positioned within a branch vessel branching from the thoracic aorta.
59 The aortic arch repair device of any one of the preceding examples wherein the plurality of first stents comprise:
first segments extending in linear manner; and
second segments connecting the first segments and positioned at apices of the first stents, wherein an indent is positioned at an interface between the first and second segments such that the second segments form a non-zero angle with the first segments.
60 The aortic arch repair device of example 53 wherein:
the stents are first stents;
the leg portion of the leg member comprises a plurality of second stents, and
wherein the first stents along the secondary lumen of the base member and the second stents along a coupling portion of the leg member are contoured to provide frictional engagement with each other along the secondary lumen.
61. The aortic arch repair device of example 60 wherein first stents are formed from nitinol wire.
62. Any of the preceding examples wherein any stent within the device has a keyhole configuration formed from nitinol wire.

### VI. Conclusion

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments can perform steps in a different order. The various embodiments described herein can also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms can also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications can be made without deviating from the technology. Further, while advantages associated with some embodiments of the technology have been described in the context of those embodiments, other embodiments can also exhibit such advantages, and not all embodiments need necessarily exhibit such advantages to fall within the scope of the technology. Accordingly, the disclosure and associated technology can encompass other embodiments not expressly shown or described herein.

1. An aortic arch repair device, comprising:
a base member having a first end portion and a second end portion, the base member comprising—
a plurality of first stents,
a plurality of second stents, and
a cover coupled to the plurality of first stents and the plurality of second stents, the cover defining a fluid conduit through the base member,
wherein
the first end portion of the base member defines a first fluid opening, and
at least a portion of the cover is positioned between the plurality of first stents and the plurality of second stents;
a septum coupled to the plurality of second stents and extending through the base member from the second end portion of the base member toward the first end portion of the base member, wherein—
the septum divides the fluid conduit into a primary lumen having a first cross-sectional area and a secondary lumen having a second cross-sectional area less than the first cross-sectional area, and
the second end portion of the base member defines a second fluid opening for the primary lumen and a third fluid opening for the secondary lumen; and
a leg member defining a leg lumen configured to be fluidly coupled to the secondary lumen,
the leg member comprising—
a coupling portion configured to be received within the secondary lumen between the septum and the cover, and
a leg portion coupled to the coupling portion and configured to extend through the third fluid opening and outwardly from the second end portion of the base member, the leg portion having a leg end portion defining a fourth fluid opening for the secondary lumen.

2. The aortic arch repair device of claim 1 wherein the plurality of first stents comprise a plurality of abluminal stents and wherein the plurality of second stents comprise a plurality of intraluminal stents.

3. The aortic arch repair device of claim 1 wherein:
the cover comprises a graft material having an outer surface and an inner surface;
the plurality of first stents are coupled to the outer surface and extend circumferentially about the outer surface, and
the plurality of second stents are coupled to the inner surface and the septum and extend circumferentially about the secondary lumen.

4. The aortic arch repair device of claim 1 wherein at least one of the plurality of first stents at least partially defines the first fluid opening and wherein at least one of the plurality of second stents at least partially defines the third fluid opening.

5. The aortic arch repair device of claim 1 wherein the leg member comprises:
a leg member cover having an outer surface and an inner surface,
a plurality of third stents positioned within the coupling portion, wherein individual ones of the plurality of third stents are coupled to the inner surface or the outer surface of the leg member cover, and
a plurality of fourth stents positioned within the leg portion, wherein individual ones of the plurality of fourth stents are coupled to the inner surface or the outer surface of the leg member cover.

6. The aortic arch repair device of claim 5 wherein one or more of the plurality of third stents are configured to be expanded when the coupling portion is received within the secondary lumen.

7. The aortic arch repair device of claim 5 wherein:
the plurality of second stents have a first cross-sectional shape comprising a first flat portion and a first curved portion,
the plurality of third stents have a second cross-sectional shape comprising a second flat portion and a second curved portion,
the second flat portion is configured to sealingly engage the first flat portion, and
the second curved portion is configured to sealingly engage the first curved portion.

8. The aortic arch repair device of claim 7, further comprising:
a pair of first markers, each of the pair of first markers positioned at a respective first intersection of the first flat portion and the first curved portion; and
a pair of second markers, each of the pair of second markers positioned at a respective second intersection of the second flat portion and the second curved portion,
wherein the pair of first markers are configured to be aligned with the pair of second markers to rotationally align the base member and the leg member.

9. The aortic arch repair device of claim 8 wherein the pair of first markers are a pair of first radiopaque markers and wherein the pair of second markers are a pair of second radiopaque markers.

10. The aortic arch repair device of claim 7, further comprising:
a first radiopaque marker at a proximal end of the leg member; and
a second radiopaque marker positioned along the septum of the base member.

11. The aortic arch repair device of claim 1 wherein none of the plurality of first stents are attached to or extend along the septum.

12. The aortic arch repair device of claim 1 wherein the coupling portion of the leg member has a first cross-sectional shape and wherein the leg portion of the leg member has a second cross-sectional shape different than the first cross-sectional shape.

13. The aortic arch repair device of claim 12 wherein the first cross-sectional shape is D-shaped and the second cross-sectional shape is circular.

14. The aortic arch repair device of claim 1 wherein:
the coupling portion of the leg member comprises a substantially D-shaped cross-section;
the leg portion of the leg member comprises a circular cross-sectional shape,
and the leg member comprises transition section comprises stents that change from the substantially D-shaped cross-section to the circular cross-section shape.

15. The aortic arch repair device of claim 14 wherein the coupling portion has a large cross-sectional dimension than the leg portion.

16. The aortic arch repair device of claim 1 wherein:
at least one of the plurality of first stents has a keyhole configuration in which linear first segments extend between second segments at apex portions of the individual first stent, the second segments forming nonzero angles relative to adjacent first segments.

17. An aortic arch repair device, comprising:
a base member having a first end portion and a second end portion, the base member comprising a plurality of stents and a cover coupled to the plurality of stents to define a fluid conduit through the base member, wherein the first end portion of the base member defines a first fluid opening;
a septum coupled to the cover and extending through the base member from the second end portion of the base member toward the first end portion of the base member,
wherein—
the septum divides the fluid conduit into a primary lumen having a first cross-sectional area and a secondary lumen having a second cross-sectional area less than the first cross-sectional area,
the secondary lumen comprises a generally flat portion adjacent the septum and a curved portion adjacent the cover, the generally flat portion being less curved than the curved portion, and
the second end portion of the base member defines a second fluid opening for the primary lumen and a third fluid opening for the secondary lumen; and
a leg member defining a leg lumen configured to be fluidly coupled to the secondary lumen,
the leg member comprising-—
a coupling portion configured to be received within the secondary lumen, the coupling portion having-—
a curved surface configured to be aligned with and sealingly engage the curved portion of the secondary lumen, and
a generally flat surface configured to be aligned with and sealingly engage the generally flat portion of the secondary lumen, the generally flat surface being less curved than the curved surface; and
a leg portion coupled to the coupling portion and configured to extend through the third fluid opening and outwardly from the second end portion of the base member, the leg portion having a leg end portion defining a fourth fluid opening for the secondary lumen.

18. The aortic arch repair device of claim 17 wherein the cover comprises a graft material having an outer surface and an inner surface, and wherein the plurality of stents comprise a plurality of first stents coupled to the outer surface of the graft material and a plurality of second stents coupled to the inner surface of the graft material.

19. The aortic arch repair device of claim 18 wherein the plurality of first stents have a first cross-sectional shape and wherein the plurality of second stents have a second cross-sectional shape different than the first cross-sectional shape.

20. The aortic arch repair device of claim 18 wherein the plurality of second stents at least partially define the secondary lumen and individual ones of the plurality of second stents comprise:
a curved section defining the curved portion of the secondary lumen, and
a generally flat section defining the generally flat portion of the secondary lumen, the generally flat section being less curved than the curved section.

21. The aortic arch repair device of claim 17 wherein the coupling portion is configured to be expanded when positioned within the secondary lumen, and wherein the expansion of the coupling portion creates the sealing engagement between the generally flat surface and the generally flat portion and the curved surface and the curved portion.

22. The aortic arch repair device of claim 17 wherein the base member extends along a first axis and the leg member extends along a second axis non-parallel to the first axis.

23. The aortic arch repair device of claim 17 wherein the base member is configured to be positioned in an ascending portion of a thoracic aorta of a patient such that the first fluid opening receives blood flow from the ascending portion of the thoracic aorta and the second fluid opening discharges the blood flow into the thoracic aorta, and wherein at least a portion of the leg member is configured to be positioned within a branch vessel branching from the thoracic aorta.

24. The aortic arch repair device of claim 17 wherein the base member is configured to be positioned in a descending portion of a thoracic aorta of a patient such that the second fluid opening receives blood flow from the thoracic aorta and the first fluid opening discharges the blood flow into the descending portion of the thoracic aorta, and wherein at least a portion of the leg member is configured to be positioned within a branch vessel branching from the thoracic aorta.

25-61. (canceled)
